SEQUENCE LISTING (65 sequences)

SEQ ID NO: 1 - DNA, 14 bp, Homo sapiens
  protein_bind (1)...(14)
aggggactttccca

SEQ ID NO: 2 - DNA, 8 bp, Homo sapiens
  protein_bind (1)...(8)
tttggcgg

SEQ ID NO: 3 - DNA, 36 bp, Homo sapiens
  protein_bind (10)...(22)
agcgtcagaaggggactttcccaagagaggccgaga

SEQ ID NO: 4 - DNA, 21 bp, Homo sapiens
  protein_bind (9)...(13)
tgaacttgtcacgctttactg

SEQ ID NO: 5 - DNA, 21 bp, Homo sapiens
  protein_bind (12)...(16)
aacttgtcacgctttactgtc

SEQ ID NO: 6 - DNA, 21 bp, Homo sapiens
  protein_bind (6)...(10)
acttgtcacgctttactgtcg

SEQ ID NO: 7 - DNA, 21 bp, Homo sapiens
  protein_bind (6)...(10)
acttgtcacgctttactgtcg

SEQ ID NO: 8 - DNA, 9 bp, Homo sapiens
  protein_bind (2)...(6)
tcgataatg

SEQ ID NO: 9 - DNA, 18 bp, Homo sapiens
  protein_bind (11)...(15)
cgataatgtgcattaagc

SEQ ID NO: 10 - DNA, 18 bp, Homo sapiens
  protein_bind (4)...(8)
cgataatgtgcattaagc

SEQ ID NO: 11 - DNA, 12 bp, Homo sapiens
  protein_bind (4)...(8)
gcattaagcaaa

SEQ ID NO: 12 - DNA, 14 bp, Homo sapiens
  protein_bind (6)...(10)
ctagttttatttgt

SEQ ID NO: 13 - DNA, 18 bp, Homo sapiens
  protein_bind (6)...(10)
agttttatttgtttattt

SEQ ID NO: 14 - DNA, 15 bp, Homo sapiens
  protein_bind (6)...(10)
agttttatttgttta

SEQ ID NO: 15 - DNA, 13 bp, Homo sapiens
  protein_bind (6)...(10)
gttttatttgttt

SEQ ID NO: 16 - DNA, 12 bp, Homo sapiens
  protein_bind (3)...(7)
ttttatttgttt

SEQ ID NO: 17 - DNA, 13 bp, Homo sapiens
  protein_bind (4)...(8)
ttttatttgttta

SEQ ID NO: 18 - DNA, 18 bp, Homo sapiens
  protein_bind (6)...(10)
ttatttgtttatttcatc

SEQ ID NO: 19 - DNA, 15 bp, Homo sapiens
  protein_bind (6)...(10)
ttatttgtttatttc

SEQ ID NO: 20 - DNA, 14 bp, Homo sapiens
  protein_bind (7)...(11)
tatttgtttatttc

SEQ ID NO: 21 - DNA, 16 bp, Homo sapiens
  protein_bind (7)...(11)
tatttgtttatttcat

SEQ ID NO: 22 - DNA, 11 bp, Homo sapiens
  protein_bind (5)...(9)
tatttgtttat

SEQ ID NO: 23 - DNA, 11 bp, Homo sapiens
  protein_bind (1)...(5)
tatttgtttat

SEQ ID NO: 24 - DNA, 14 bp, Homo sapiens
  protein_bind (6)...(10)
atttgtttatttca

SEQ ID NO: 25 - DNA, 13 bp, Homo sapiens
  protein_bind (4)...(8)
atttgtttatttc

SEQ ID NO: 26 - DNA, 13 bp, Homo sapiens
  protein_bind (4)...(8)
atttgtttatttc

SEQ ID NO: 27 - DNA, 12 bp, Homo sapiens
  protein_bind (5)...(9)
atttgtttattt

SEQ ID NO: 28 - DNA, 12 bp, Homo sapiens
  protein_bind (3)...(7)
atttgtttattt

SEQ ID NO: 29 - DNA, 11 bp, Homo sapiens
  protein_bind (1)...(5)
tgtttatttca

SEQ ID NO: 30 - DNA, 18 bp, Homo sapiens
  protein_bind (12)...(16)
gtttatttcatcttctaa

SEQ ID NO: 31 - DNA, 12 bp, Homo sapiens
  protein_bind (5)...(9)
ttctaagtataa

SEQ ID NO: 32 - DNA, 24 bp, Homo sapiens
  protein_bind (6)...(10)
ttctaagtataagaatacattgta

SEQ ID NO: 33 - DNA, 13 bp, Homo sapiens
  protein_bind (5)...(9)
agcattcccacca

SEQ ID NO: 34 - DNA, 13 bp, Homo sapiens
  protein_bind (5)...(9)
agcattcccacca

SEQ ID NO: 35 - DNA, 8 bp, Homo sapiens
  protein_bind (2)...(6)
gcgacaaa

SEQ ID NO: 36 - DNA, 21 bp, Homo sapiens
  protein_bind (12)...(16)
agccctgcgctccttacgaca

SEQ ID NO: 37 - DNA, 10 bp, Homo sapiens
  protein_bind (5)...(9)
gcctcgtgcg

SEQ ID NO: 38 - DNA, 12 bp, Homo sapiens
  protein_bind (6)...(10)
gcgggccaatcg

SEQ ID NO: 39 - DNA, 21 bp, Homo sapiens
  protein_bind (12)...(16)
cgctgctcccgggtgatgacg

SEQ ID NO: 40 - DNA, 21 bp, Homo sapiens
  protein_bind (11)...(15)
tgctcccgggtgatgacgtag

SEQ ID NO: 41 - DNA, 19 bp, Homo sapiens
  protein_bind (5)...(9)
gggtgatgacgtaggctgc

SEQ ID NO: 42 - DNA, 12 bp, Homo sapiens
  protein_bind (5)...(9)
gtgatgacgtag

SEQ ID NO: 43 - DNA, 12 bp, Homo sapiens
  protein_bind (3)...(7)
gatgacgtaggc

SEQ ID NO: 44 - DNA, 12 bp, Homo sapiens
  protein_bind (3)...(7)
gatgacgtaggc

SEQ ID NO: 45 - DNA, 9 bp, Homo sapiens
  protein_bind (5)...(9)
tgacgtagg

SEQ ID NO: 46 - DNA, 8 bp, Homo sapiens
  protein_bind (1)...(5)
tgacgtag

SEQ ID NO: 47 - DNA, 8 bp, Homo sapiens
  protein_bind (2)...(6)
agggaggg

SEQ ID NO: 48 - DNA, 8 bp, Homo sapiens
  protein_bind (2)...(6)
cttggcgg

SEQ ID NO: 49 - DNA, 8 bp, Homo sapiens
  protein_bind (3)...(7)
cttggcgg

SEQ ID NO: 50 - DNA, 8 bp, Homo sapiens
  protein_bind (3)...(7)
cttggcgg

SEQ ID NO: 51 - DNA, 43 bp, Homo sapiens
tgaacttgtcacgctttactgtcgataatgtgcattaagcaaa

SEQ ID NO: 52 - DNA, 48 bp, Homo sapiens
ctagttttatttgtttatttcatcttctaagtataagaatacattgta

SEQ ID NO: 53 - DNA, 13 bp, Homo sapiens
agcattcccacca

SEQ ID NO: 54 - DNA, 28 bp, Homo sapiens
gcgacaaagccctgcgctccttacgaca

SEQ ID NO: 55 - DNA, 10 bp, Homo sapiens
gcctcgtgcg

SEQ ID NO: 56 - DNA, 40 bp, Homo sapiens
gcgggcaatcgcgctgctcccgggtgatgacgtaggctgc

SEQ ID NO: 57 - DNA, 8 bp, Homo sapiens
agggaggg

SEQ ID NO: 58 - DNA, 8 bp, Homo sapiens
cttggcgg

SEQ ID NO: 59 - DNA, 2320 bp, Homo sapiens
gcgcagggaggggagaccttggcggacggcggagccccagcggaggtgaaagtattggcggaaaggaaaatacagcggaaaaatgcagagctggagtcgtgtgtactgctccttggccaagagaggccatttcaatcgaatatctcatggcctacagggactttctgcagtgcctctgagaacttacgcagatcagccgattgatgctgatgtaacagttataggttctggtcctggaggatatgttgctgctattaaagctgcccagttaggcttcaagacagtctgcattgagaaaaatgaaacacttggtggaacatgcttgaatgttggttgtattccttctaaggctttattgaacaactctcattattaccatatggcccatggaacagattttgcatctagaggaattgaaatgtccgaagttcgcttgaatttagacaagatgatggagcagaagagtactgcagtaaaagctttaacaggtggaattgcccacttattcaaacagaataaggttgttcatgtcaatggatatggaaagataactggcaaaaatcaagtcactgctacgaaagctgatggcggcactcaggttattgatacaaagaacattcttatagccacgggttcagaagttactccttttcctggaatcacgatagatgaagatacaatagtgtcatctacaggtgctttatctttaaaaaaagttccagaaaagatggttgttattggtgcaggagtaataggtgtagaattgggttcagtttggcaaagacttggtgcagatgtgacagcagttgaatttttaggtcatgtaggtggagttggaattgatatggagatatctaaaaactttcaacgcatccttcaaaaacaggggtttaaatttaaattgaatacaaaggttactggtgctaccaagaagtcagatggaaaaattgatgtttctattgaagctgcttctggtggtaaagctgaagttatcacttgtgatgtactcttggtttgcattggccgacgaccctttactaagaatttgggactagaagagctgggaattgaactagatcctagaggtagaattccagtcaataccagatttcaaactaaaattccaaatatctatgccattggtgatgtagttgctggtccaatgctggctcacaaagcagaggatgaaggcattatctgtgttgaaggaatggctggtggtgctgtgcacattgactacaattgtgtgccatcagtgatttacacacaccctgaagttgcttgggttggcaaatcagaagagcagttgaaagaagagggtattgagtacaaagttgggaaattcccatttgctgctaacagcagagctaagacaaatgctgacacagatggcatggtgaagatccttgggcagaaatcgacagacagagtactgggagcacatattcttggaccaggtgctggagaaatggtaaatgaagctgctcttgctttggaatatggagcatcctgtgaagatatagctagagtctgtcatgcacatccgaccttatcagaagcttttagagaagcaaatcttgctgcgtcatttggcaaatcaatcaacttttgaattagaagattatatatttttttttctgaaatttcctgggagcttttgtagaagtcacattcctgaacaggatattctcacagctccaagaatttctaggactgaattatgaaacttttggaaggtatttaataggtttggacaaaatggaatactcttatatctatattttacataaatttagtattttgtttcagtgcactaatatgtaagacaaaaaggactacttattgtagtcatcctggaatatctccgtcaactcatattttcatgctgttcatgaaagattcaatgcccctgaatttaaatagctcttttctctgatacagaaaagttgaattttacatggctggagctagaatttgatatgtgaacagttgtgtttgaagcacagtgatcaagttatttttaatttggttttcacattggaaacaagtcagtcattcagatatgattcaaatgtctataaaccaaactgatgtaagtaaatggtctctcacttgttttatttaacctctaaattctttcattttaggggtagcatttgtgttgaagaggttttaaagcttccattgttgtctgcaactctgaagggtaattatatagttacccaaattaagagagtctatttacggaactcaaatacgtgggcattcaaatgtattacagtggggaatgaagatactgaaataaacgtcttaaatattc

SEQ ID NO: 60 - DNA, 2108 bp, Homo sapiens
ggcacgagggaggcgcccagcggaggtgaaagtattggcggaaaggaaaatacagcggaaaaatgcagagctggagtcgtgtgtactgctccttggccaagagaggccatttcaatcgaatatctcatggcctacagggactttctgcagtgcctctgagaacttacgcagatcagccgattgatgctgatgtaacagttataggttctggtcctggaggatatgttgctgctattaaagctgcccagttaggcttcaagacagtctgcattgagaaaaatgaaacacttggtggaacatgcttgaatgttggttgtattccttctaaggctttattgaacaactctcattattaccatatggcccatggaaaagattttgcatctagaggaattgaaatgtccgaagttcgcttgaatttagacaagatgatggagcagaagagtactgcagtaaaagctttaacaggtggaattgcccacttattcaaacagaataaggttgttcatgtcaatggatatggaaagataactggcaaaaatcaagtcactgctacgaaagctgatggcggcactcaggttattgatacaaagaacattcttatagccacgggttcagaagttactccttttcctggaatcacgatagatgaagatacaatagtgtcatctacaggtgctttatctttaaaaaaagttccagaaaagatggttgttattggtgcaggagtaataggtgtagaattgggttcagtttggcaaagacttggtgcagatgtgacagcagttgaatttttaggtcatgtaggtggagttggaattgatatggagatatctaaaaactttcaacgcatccttcaaaaacaggggtttaaatttaaattgaatacaaaggttactggtgctaccaagaagtcagatggaaaaattgatgtttctattgaagctgcttctggtggtaaagctgaagttatcacttgtgatgtactcttggtttgcattggccgacgaccctttactaagaatttgggactagaagagctgggaattgaactagatcccagaggtagaattccagtcaataccagatttcaaactaaaattccaaatatctatgccattggtgatgtagttgctggtccaatgctggctcacaaagcagaggatgaaggcattatctgtgttgaaggaatggctggtggtgctgtgcacattgactacaattgtgtgccatcagtgatttacacacaccctgaagttgcttgggttggcaaatcagaagagcagttgaaagaagagggtattgagtacaaagttgggaaattcccatttgctgctaacagcagagctaagacaaatgctgacacagatggcatggtgaagatccttgggcagaaatcgacagacagagtactgggagcacatattcttggaccaggtgctggagaaatggtaaatgaagctgctcttgctttggaatatggagcatcctgtgaagatatagctagagtctgtcatgcacatccgaccttatcagaagcttttagagaagcaaatcttgctgcgtcatttggcaaatcaatcaacttttgaattagaagattatatatatttttttctgaaatttcctgggagcttttgtagaagtcacattcctgaacaggatattctcacagctccaagaatttctaggactgaattatgaaacttttggaaggtatttaataggtttggacaaaatggaatactcttatatctatattttacataaatttagtattttgtttcagtgcactaatgtgtaagacaaaaagctacttattgtagcatcctggaatatctccgtcaactcatattttcatgctgttcatgaaagattcaatgcccctgaatttaaatagcttttttctctgatacagaaaagttgaattttacatggctggagctagaatttgatatgtgaacagttgtgtttgaagcacagtgatcaagttatttttaatttggttttcacattggaaacaagtcagtcattcagatatgattcaaatgtctataaaccgaactgatgtaagtaaaaaaaaaaaaaaaaaaaaaaaaaaaaaaaaa

SEQ ID NO: 61 - DNA, 2341 bp, Homo sapiens
acgtaggctgcgcctgtgcatgcgcagggaggggagaccttggcggacggcggagccccagcggaggtgaaagtattggcggaaaggaaaatacagcggaaaaatgcagagctggagtcgtgtgtactgctccttggccaagagaggccatttcaatcgaatatctcatggcctacagggactttctgcagtgcctctgagaacttacgcagatcagccgattgatgctgatgtaacagttataggttctggtcctggaggatatgttgctgctattaaagctgcccagttaggcttcaagacagtctgcattgagaaaaatgaaacacttggtggaacatgcttgaatgttggttgtattccttctaaggctttattgaacaactctcattattaccatatggcccatggaacagattttgcatctagaggaattgaaatgtccgaagttcgcttgaatttagacaagatgatggagcagaagagtactgcagtaaaagctttaacaggtggaattgcccacttattcaaacagaataaggttgttcatgtcaatggatatggaaagataactggcaaaaatcaagtcactgctacgaaagctgatggcggcactcaggttattgatacaaagaacattcttatagccacgggttcagaagttactccttttcctggaatcacgatagatgaagatacaatagtgtcatctacaggtgctttatctttaaaaaaagttccagaaaagatggttgttattggtgcaggagtaataggtgtagaattgggttcagtttggcaaagacttggtgcagatgtgacagcagttgaatttttaggtcatgtaggtggagttggaattgatatggagatatctaaaaactttcaacgcatccttcaaaaacaggggtttaaatttaaattgaatacaaaggttactggtgctaccaagaagtcagatggaaaaattgatgtttctattgaagctgcttctggtggtaaagctgaagttatcacttgtgatgtactcttggtttgcattggccgacgaccctttactaagaatttgggactagaagagctgggaattgaactagatcctagaggtagaattccagtcaataccagatttcaaactaaaattccaaatatctatgccattggtgatgtagttgctggtccaatgctggctcacaaagcagaggatgaaggcattatctgtgttgaaggaatggctggtggtgctgtgcacattgactacaattgtgtgccatcagtgatttacacacaccctgaagttgcttgggttggcaaatcagaagagcagttgaaagaagagggtattgagtacaaagttgggaaattcccatttgctgctaacagcagagctaagacaaatgctgacacagatggcatggtgaagatccttgggcagaaatcgacagacagagtactgggagcacatattcttggaccaggtgctggagaaatggtaaatgaagctgctcttgctttggaatatggagcatcctgtgaagatatagctagagtctgtcatgcacatccgaccttatcagaagcttttagagaagcaaatcttgctgcgtcatttggcaaatcaatcaacttttgaattagaagattatatatttttttttctgaaatttcctgggagcttttgtagaagtcacattcctgaacaggatattctcacagctccaagaatttctaggactgaattatgaaacttttggaaggtatttaataggtttggacaaaatggaatactcttatatctatattttacataaatttagtattttgtttcagtgcactaatatgtaagacaaaaaggactacttattgtagtcatcctggaatatctccgtcaactcatattttcatgctgttcatgaaagattcaatgcccctgaatttaaatagctcttttctctgatacagaaaagttgaattttacatggctggagctagaatttgatatgtgaacagttgtgtttgaagcacagtgatcaagttatttttaatttggttttcacattggaaacaagtcagtcattcagatatgattcaaatgtctataaaccaaactgatgtaagtaaatggtctctcacttgttttatttaacctctaaattctttcattttaggggtagcatttgtgttgaagaggttttaaagcttccattgttgtctgcaactctgaagggtaattatatagttacccaaattaagagagtctatttacggaactcaaatacgtgggcattcaaatgtattacagtggggaatgaagatactgaaataaacgtcttaaatattc

SEQ ID NO: 62 - DNA, 301 bp, Homo sapiens
gttgaacttgtcacgctttactgtcgataatgtgcattaagcaaacgctagttttatttgtttatttcatcttctaagtataagaatacattgtagctcgacattttggcaccagcccctaaagcattcccaccaccacccccgctgcgacaaagccctgcgctccttacgacagcgtacgacgccgagcctgacaggaacgcctcgtgcggtagaaccgcgcgggccaatcgcgctgctcccgggtgatgacgtaggctgcgcctgtgcatgcgcagggaggggagaccttggcggagcg

SEQ ID NO: 63 - DNA, 21 bp, Homo sapiens
  protein_bind (9)...(13)
tgaacttatcacgctttactg

SEQ ID NO: 64 - DNA, 18 bp, Homo sapiens
  protein_bind (4)...(8)
caataatgtgcattaagc

SEQ ID NO: 65 - DNA, 12 bp, Homo sapiens
  protein_bind (5)...(8)
agcattccacca

(19) United States
(12) Patent Application Publication
(10) Pub. No.: US 2004/0191781 A1
(43) Pub. Date: Sep. 30, 2004

(54) Genomic profiling of regulatory factor binding sites

(76) Inventors: Jie Zhang, Campbell, CA (US); Hsiu-Ying Wei, San Jose, CA (US); Leslie Margaret McEvoy, Mountain View, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/402,689

(22) Filed: Mar. 28, 2003

(51) Int. Cl. C12Q 1/68; G06F 19/00; G01N 33/48; G01N 33/50

(52) U.S. Cl. 435/6; 702/20

(57) ABSTRACT

A method is provided for profiling regulatory factor binding sites. A complete gene is located on genome for mapping gene regulatory regions. Genomic sequences of gene regulatory regions are defined and retrieved. DNA sequence information of each retrieved gene regulatory region is screened for identifying putative regulatory factor binding sites. The putative regulatory factor binding sites are profiled.

### CROSS-REFERENCE TO RELATED APPLICATIONS

This application is related to U.S. Ser. No. ______, filed ______, entitled “Statistical Analysis of Regulatory Factor Binding of Differentially Expressed Genes”, and identified as Attorney Docket No. 39753-0002, which application is fully incorporated herein.

### BACKGROUND OF THE INVENTION

1. Field of the Invention

The present invention relates generally to methods, systems and data structures that provide profiles of regulatory factor binding sites of all the known genes, and more particularly to methods, data structures and systems for identifying and characterizing regulatory factors binding sites in order to develop statistic analysis on identified binding sites for further therapeutic strategies development.

2. Description of the Related Art

Altering gene expression level has become an important and efficient approach to address the human disorders. The expression level of each gene is controlled by the transcription machinery, in which some specific proteins called transcription factors (TFs) binds to the regulatory region of the gene, and in turn to initialize the transcription processes. Thus, the corresponding TFs and their binding sites on gene regulatory region can play the essential role in controlling the expression level of gene. Therefore, transcription factors and their related transcription mechanisms have become the “hot” spots in modern bio-medical research and development efforts.

For each gene, the transcription starting site (TSS) is the position where its' mRNA starts to be transcribed from DNA by RNA polymerase II. During this process, the gene regulatory region is associated and bound by certain regulatory factors. These bound factors together with other transcription proteins formed a transcription complex that can initialize the transcription process. More specifically, this typically includes the transcription factor binding sites that are the short consensus genomic sequences that locate immediately before the transcription start sites of genes. A transcription regulatory region can contain several binding sites, therefore can be bound by several corresponding transcription factors. All the necessary transcription factors needs to bind to the 5 prime region of TSS to initiate the transcription machinery. Thus, identifying TSS is important to define the transcription regulatory region for each gene. Currently, many specific researches and developments focus their efforts on the specific TFs and corresponding binding sites, which provided many solid data but still failed to meet the large requirement of the development of genomic-related biomedical needs. To meet the fast growing transcription factors related drug discovery business and challenge, it is very important to identify all putative regulatory factors and characterize their corresponding binding sites in genome. Especially, with the finish of human genome project and occurrences of large amount of disease-related gene expression data (such as microarray-based data), the genome wide profiling of regulatory factor binding sites become urgent.

The present invention retrieved all the full-length genes from various public available databases (such as, NCBI refseq, NIH MGC consortium, DBTSS database of Japan, and so on) and then mapped these gene's TSSs on the most updated Human Genome Working Draft (such as Assembly version June, 2002, Kent et al., Genome Res. 12, 996). Then it defined the most 5 prime TSS for each gene by comparing all the possible TSSs generated by mapping of this gene. The transcription regulatory region (TRR), such as core promoter regions were defined based on the most 5′ TSS positions, and their corresponding genomic sequences were retrieved from most updated human genome for further analysis. The profiled TRR for all the known genes were stored in a database for further drug-target related statistic analysis and for further therapeutic strategies development.

### SUMMARY OF THE INVENTION

Accordingly, an object of the present invention is to provide improved methods for genomic-profiling regulatory factor binding sites, as well as data structures and systems associated with the methods.

In another object of the present invention, methods for profiling regulatory factor binding sites, as well as data structures and systems associated with the methods, are provided that employ genome-wide probability mapping relative to profiled binding sites.

Yet another object of the present invention is to provide improved methods for biomedical research, as well as data structures and systems associated with the methods.

A further object of the present invention is to provide improved methods for pre-clinical development, as well as data structures and systems associated with the methods.

Still another object of the present invention is to provide improved methods for drug screening applications, as well as data structures and systems associated with the methods.

Another object of the present invention is to provide improved methods for target discovering and target validation, as well as data structures and systems associated with the methods.

Yet another object of the present invention is to provide improved methods for profiling of a regulatory region, as well as data structures and systems associated with the methods.

A further object of the present invention is to provide improved methods for building the genome or tissue wide connections between regulatory profilings of different genes, as well as data structures and systems associated with the methods.

Still a further object of the present invention is to provide improved methods for understanding the genome or tissue or cell background of various known transcription profiling understanding the genome or tissue or cell background of various known transcription profiling, as well as data structures and systems associated with the methods.

These and other objects of the present invention are achieved in a method for profiling regulatory factor binding sites. A complete gene is located on genome for mapping gene regulatory regions. Genomic sequences of gene regulatory regions are defined and retrieved. DNA sequence information of each retrieved gene regulatory region is screened for identifying putative regulatory factor binding sites. The putative regulatory factor binding sites are profiled.

In another embodiment of the present invention, a method for profiling identified binding sites provides a database that includes profiled identified binding sites for all known genes. Probability statistic analysis is applied to the profiled binding sites.

In another embodiment of the present invention, a data structure tangibly stored on a computer readable medium is provided. The data structure includes a database with profiled identified binding sites. The profiled identified binding sites are created by screening DNA sequence information of gene regulatory regions. The database is searchable by gene identifiers.

In another embodiment of the present invention, a computer implemented system for displaying profiled regulatory factor binding sites includes a database that includes profiled identified binding sites. The profiled identified binding sites are created by screening DNA sequence information of gene regulatory regions. The database is searchable by gene identifiers. A user interface is provided that includes one or more selectable user inputs. An input device is operable by a user. A display is included that displays at least one output in response to the profiled identified binding sites.

### BRIEF DESCRIPTION OF THE DRAWINGS

FIG. 1 is a flow chart illustrating one of the embodiment of the present invention for profiling regulatory factor binding sites.

FIG. 2 is a flow chart that described how to define the transcription regulatory region of a gene (example Gene X).

FIG. 3 is a flow chart illustrating calculating the frequency of TF binding sites.

FIG. 4 illustrates that the core promoter region can include 200-300 bases upstream and about 50-100 bases downstream of the TSS.

FIG. 5 is a description of one embodiment of a structure of a database of the present invention.

FIG. 6 is a flow chart illustrating the FIG. 5 database.

FIG. 7 lists the complete sequences for gene DLD retrieved from the refseq database (SEQ ID NO 59).

FIG. 8 lists the complete sequences for gene DLD retrieved from the MGC database (SEQ ID NO 60).

FIG. 9 lists the complete sequences for gene DLD retrieved from the DBTSS database (SEQ ID NO 61).

FIG. 10 lists the stored sequence for gene DLD (SEQ ID NO 62).

FIG. 11 is a screen shot of a query form that can be used with the FIG. 7 database.

FIG. 12 is a screen shot of one embodiment of a database query result from the FIG. 5 database.

FIG. 13 illustrates one embodiment of a system of the present invention.

### DESCRIPTION OF THE PREFERRED EMBODIMENTS

In various embodiments, the present invention provides methods for genome wide profiling regulatory factor binder sites, data structures tangibly stored on a computer readable medium, and associated systems. Examples of regulatory factor binder sites include but are not limited to, sequence AGGGGACTTTCCCA (SEQ ID NO 1) as the binding sites for transcription factor NF-kappa B; sequence TTTGGCGG (SEQ ID NO 2) as the binding sites for transcription factor E2F-1, and the like.

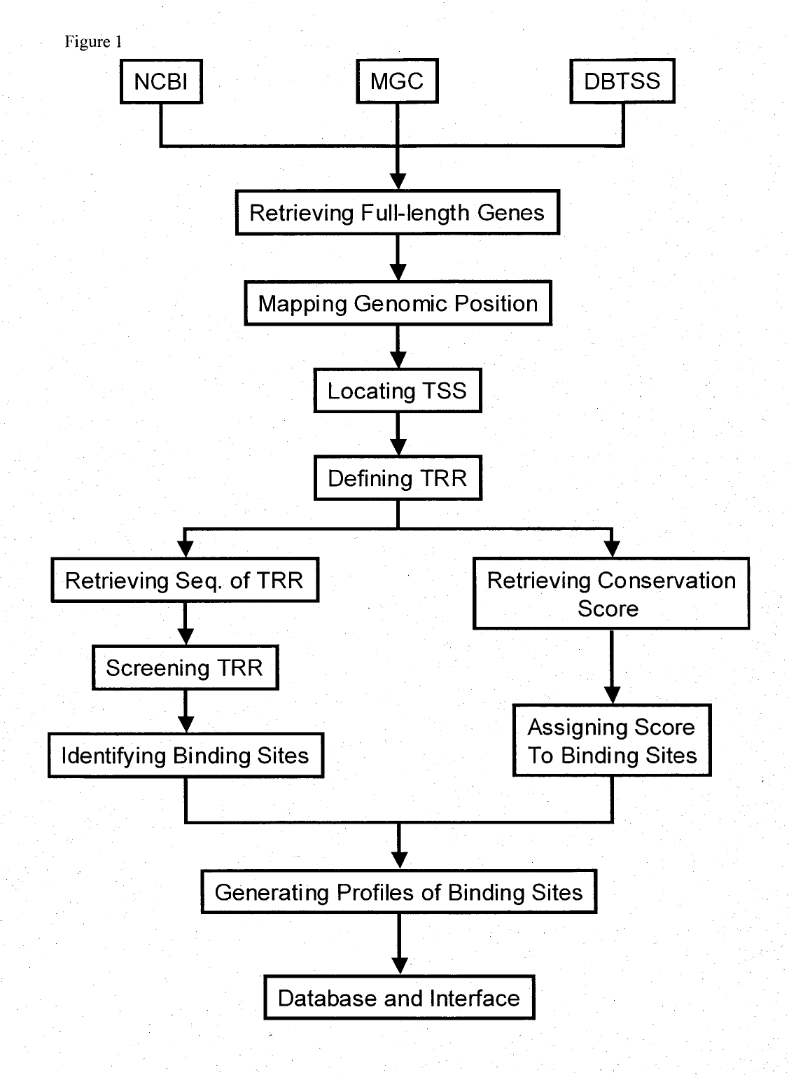

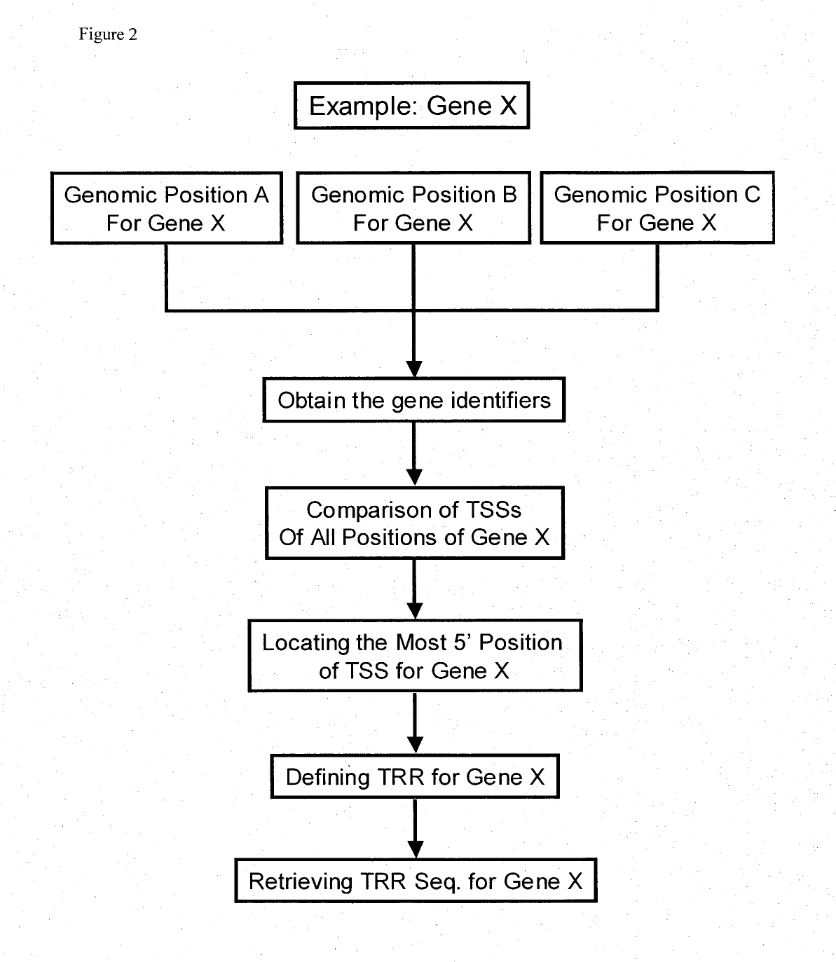

Referring to the flow charts of FIGS. 1 and 2, in one embodiment of the present invention, genomic sequences of gene regulatory regions are retrieved and are mapped to human genome. Based on the mapped genes, the most 5 prime position of TSS for each gene is identified and the corresponding regulatory region for the gene is identified. DNA sequence information for each retrieved gene regulatory region is screened to identify putative regulatory factor binding sites. The putative regulatory factor binding sites are then profiled.

Information retrieved from the database can be utilized for a variety of different purposes and applications including but not limited to, biomedical research, pre-clinical development, drug screening applications, target discovering and target validation, profiling of a regulatory region, building the genome or tissue wide connections between regulatory profilings of different genes, understanding the genome or tissue background of various known transcription profiling understanding the genome or tissue background of various known transcription profiling, and the like.

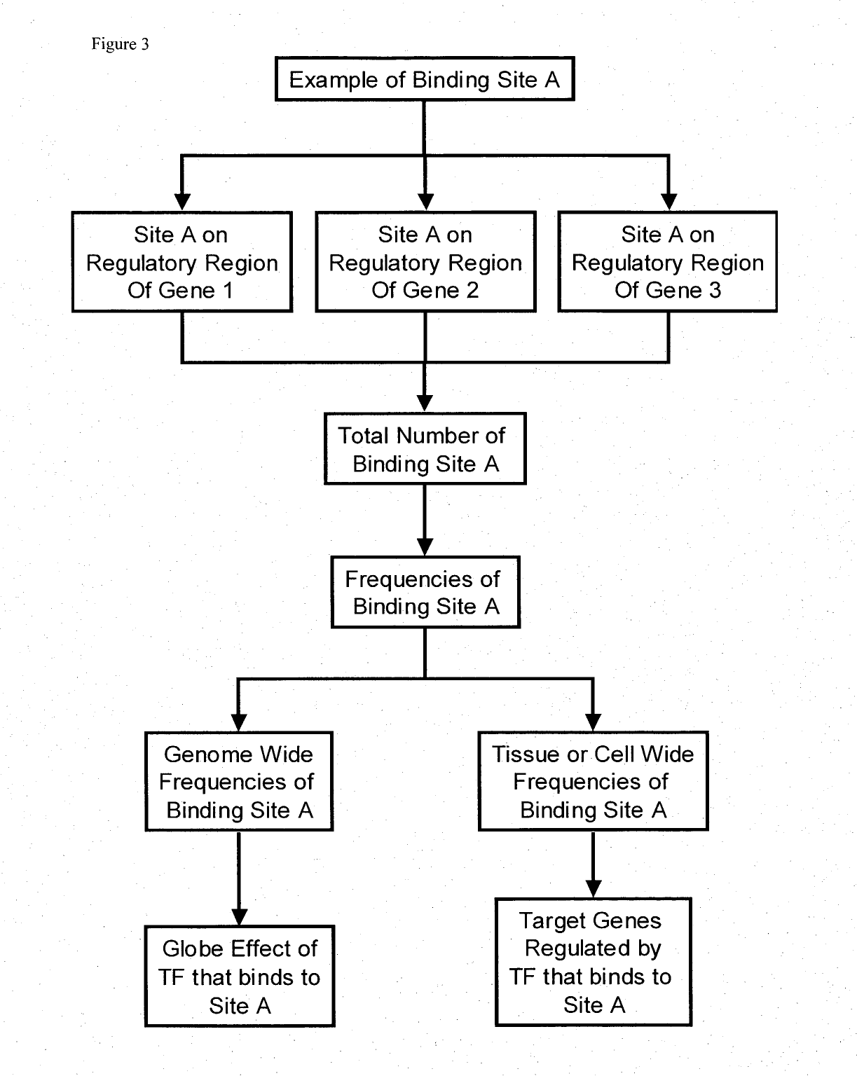

Referring to FIG. 3, probability mapping is applied to the identified binding sites. The probability mapping describes the identification of existences of a specific transcription regulatory factor binding sites, such as all the putative E2F-1 sites, in the regulatory region of all the genes or in the genes that expressed in certain tissue or cell. The probability mapping tells how many genes are possibly transcription-regulated by a specific regulatory factor. It also indicates that how much globe, genome wide, cell wide, or tissue wide, effect a specific regulator factor could have. This information is very useful for bio-medical research based therapeutic method development.

In another embodiment of the present invention a full length gene is mapped for purposes of mapping gene regulatory regions. It will be appreciated that for purposes of this specification, full length extends to the length of the gene. This can cause a slight shift of the genomic position of the transcription start sites of the different versions of the same gene. In one embodiment, all of the available full-length gene is used in a comparision in order to obtain the most 5′ TSS. Based on the most 5′ TSS, the regulatory regions of genes are defined and the genomic sequences of gene regulatory regions are retrieved. DNA sequence information is screened for each retrieved gene regulatory region to identify putative regulatory factor binding sites. The putative regulatory factor binding sites are mapped to the human genome.

Full-length genes are retrieved to provide sequences information for retrieved genes. The retrieved genes can be mapped to a recently updated human genome using a tool provided by a public available UCSC genome browser databases, self-developed scripts, and the like. In one embodiment, the transcription start site is mapped. In one embodiment, the TSS is mapped by taking the most 5′ TSS of each gene after comparing all available TSS's for the gene, illustrated in FIG. 2.

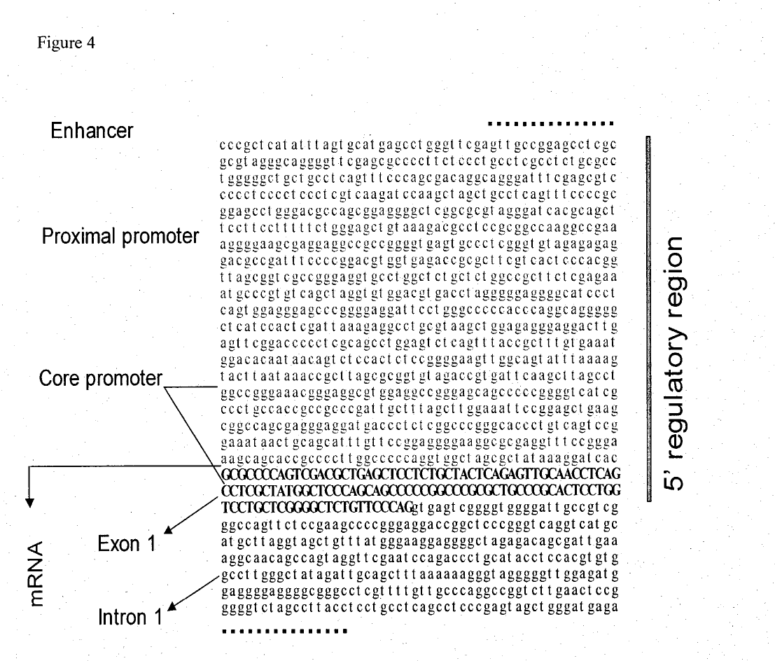

A genomic sequence of a regulatory region can be retrieved for each retrieved gene with the most 5′ TSS from the most updated human genome. The 5′ regulatory region is the sequences upstream of the TSS and downstream of the TSS. In various embodiments, the gene regulatory regions include but are not limit to, the core promoter region, the upstream enhancer region, a downstream regulatory region, and the like, as illustrated in FIG. 4. The core promoter region can include 200-300 bases upstream and about 50-100 bases downstream of the TSS.

Corresponding sequences relative to TSS can be cut and stored. The corresponding sequences relative to TSS can be cut and stored with the use of self-developed scripts from genomic sequences based on a specific release, older, updated and future releases, including but not limited to the UCSC genome browser, NCBI genome database, the Ensembl database, other genomic sequence databases and the like.

In one embodiment, the DNA sequence information is screened using a MATCH program that is licensed from TRANSFAC database. The DNA sequence information screening can include selecting the TF matrix, scores of matrix similarity, scores of core similarity, and the like.

Cut-off is applied to reduce the false positive and false negative matching during screening. A genomic or tissue-specific frequency of each binding site and can be determined. The frequency can be the existence of specific TF binding sites in regulatory regions of at least one of, (i) all the genes genome wide, (ii) all the genes specific cell wide, (iii) all the genes specific-tissue wide, (iv) all the genes specific-defined. The frequency can be the existence of specific TF binding sites in regulatory regions of tissue specific genes. Additionally, the frequency can also be considered with a conservation score or an expression level score. By way of illustration, and without limitation, the identified binding sites can be considered differently based on their corresponding conservation score or their corresponding gene expression level. For example, a binding site with higher conservation score or the corresponding gene with higher expression level could play a more significant role than those with lower scores.

The conservation score for each binding site can be created. The conservation score is selected to cover regions where the TF binding sites are identified as well as any other measurements that indicate conservation levels between the two species including but not limited to mouse and human. The position of each binding site can be determined. The position can be based on a human genome working draft. The position is a converted position in a human genome working draft. As more sequence pieces are added, the total length for each chromosome grows. This shifts the position reading for each base on the chromosome. However, the position can be easily converted and the relative position of a regulatory region to the position of the gene remains unchanged. The genome position of a start and end can be determined. A distance of each binding site to the TSS can be determined. The distance is relative to a number of bases between a binding site and the TSS. By way of illustration, and without limitation, in one embodiment the distance is that of the last base between defined binding sites to the base of TSS's 23 base. In this example, there are 23 bases between these two specific bases.

In one embodiment of the present invention, based on the positions of most 5 prime TSSs, the 5 prime regulatory sequences from most updated Human Genome Working Draft are retrieved for all the available genes using self developed computer scripts and programs.

These retrieved sequences include but not limited to 250-base 5 prime upstream and 50-base 3 prime downstream of TSS for each gene.

All the regulatory region sequences can be analyzed using well-characterized transcription factor binding consensus sequence patterns (or, position weighted matrix) created by licensed TRANSFAC databases (TRANSFAC professional 6.3 version, Wingender et al., Nucleic Acids Res. 29, 281). The sites with high score matching with binding matrix will be selected. These sites include their positions in the genome (relative to specific genome assemble version) and their lengths and their synergism information with flanking sites.

All the binding sites result from above are further analyzed by comparing their conservation scores with mouse. The mouse genome and relative conservation information will be retrieved from public available NCBI and UCSC genome databases, and the conservation comparison with human transcription factor binding sites will be done using self-generated scripts and programs.

The resulted transcription factor binding site sequences information from above, include their genomic positions (start, end), length, distant to TSS of each gene, and the flanking regions (include but not limit to 10-base both 5 prime and 3 prime) will be deposited into a database. The related reference links such as gene name, function, annotation, et al are also added.

All the possible transcription decoys can be computational generated based on the database. The decoys can further be experimentally screened by using high-throughput methods, such as oligo-array, capillary-electrophoresis, et al for binding efficiency optimization. All the optimized decoy information will be deposited into the database. The partial information in the database can be used in future versions of the database.

Profiles of the regulatory regions of genes include but are not limited to, (i) probability mapping of each regulatory factor binding site, (ii) target genes identification for each known regulatory factor; (iii) statistic analysis of regulatory factor binding profiles of genes identified from various differential expressed genes, and the like.

In one embodiment, a length of each binding site is determined. Sequence information about regions adjacent to the binding site can also be determined. Again by illustration and without limitation, one example is agcgtcagaAGGGGACTTTCCCaagagaggccgaga, (SEQ ID NO 3) with the small case base letters flanking the core binding sites, in upper case.

Co-existence information of other binding sites can also be ascertained. The transcription machinery usually requires the formation of the complex by several different transcription related proteins and includes the several different DNA binding factors. When the present invention, the binding sites are profiled for a regulatory region of the gene and often more than one binding site is identified from a single region. The number of binding sites can be, by way of example, fifteen to twenty from a single region. The cluster of the binding sites and their positions can be determined.

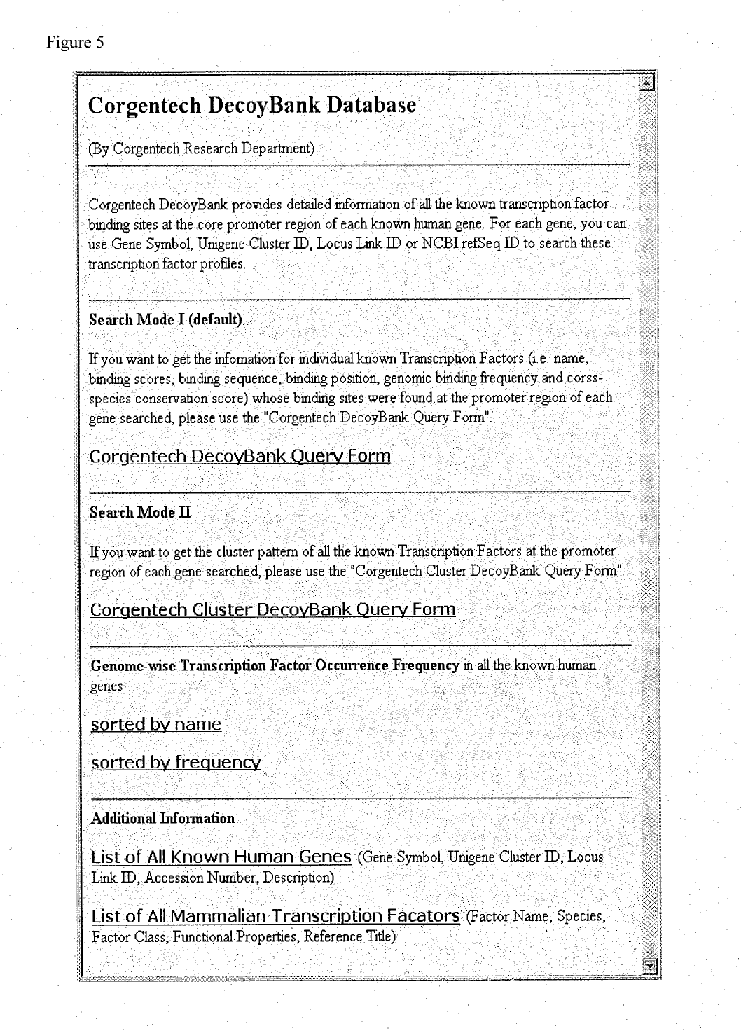

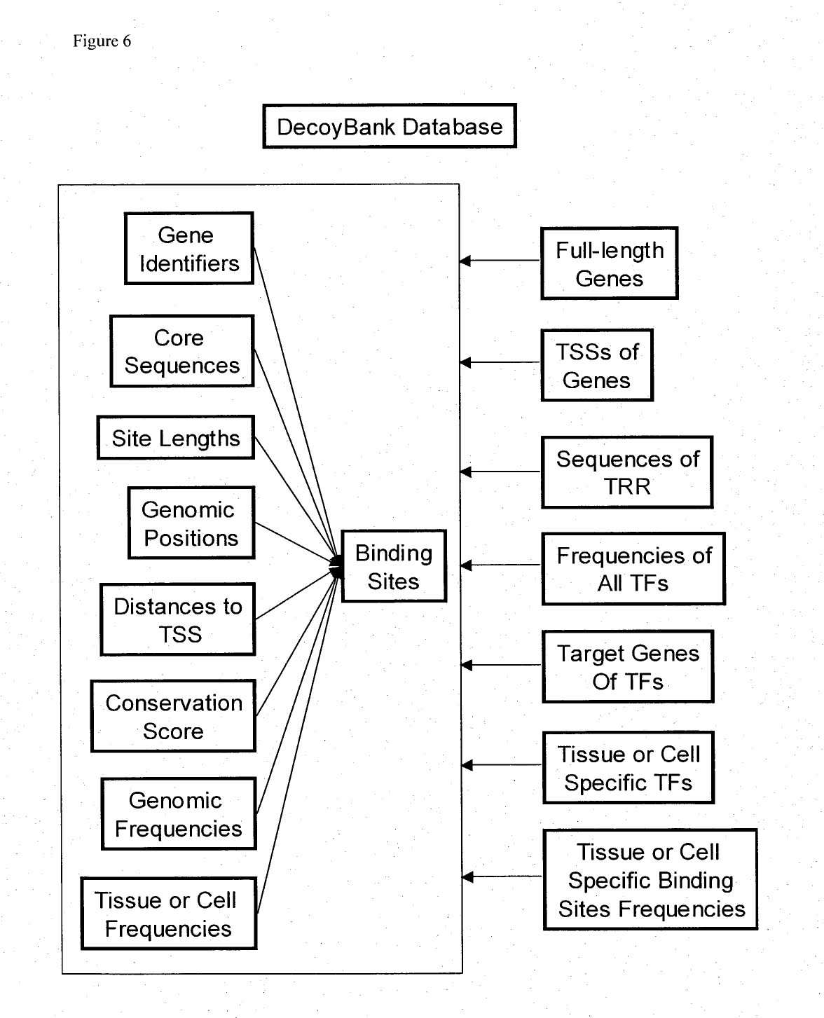

Referring now to FIGS. 5 and 6, another embodiment of the present invention is a data structure tangibly stored on a computer readable medium that includes a database with the profiled identified binding site information. The database includes a core table with identifiers, binding sites and the like. Binding site information includes but is not limited to, sequence, length, position, direction, frequency, and the like. One supporting table includes TSS position of all genes. A sequence table provides the sequences of regulatory regions of genes. Additional support tables include but are not limited to frequency of TF, target genes of TF for each TF, and the like.

All of the tables are linked by one or more identifiers. In one embodiment, several instead of one perl CGI script are used to reach and search the database and then display the corresponding information. A web-browser interface is provided.

The database is searchable by a variety of different means, including but not limited to gene identifiers, gene symbol, or self-developed identifiers and the like. Gene identifiers can be selected from the NCBI database, which can be a, Unigene Cluster ID, LoucsLink ID, international approved gene symbols, and the like.

In one embodiment, the database includes genomic frequencies information for TF, and is sortable by at least a TF name or TF frequencies. The TF frequencies can include genome frequencies and tissue specific frequencies. In one specific example, the database contains the profiles of regulatory factor binding sites for all the known genes (about 15,450 total).

By way of illustration, and without limitation, one gene (symbol: DLD, dihydrolipoamide dehydrogenase) is used to briefly show how the database is built.

1. Retrieving of Full Length Genes for an Example Gene DLD to Provide Sequences Information.

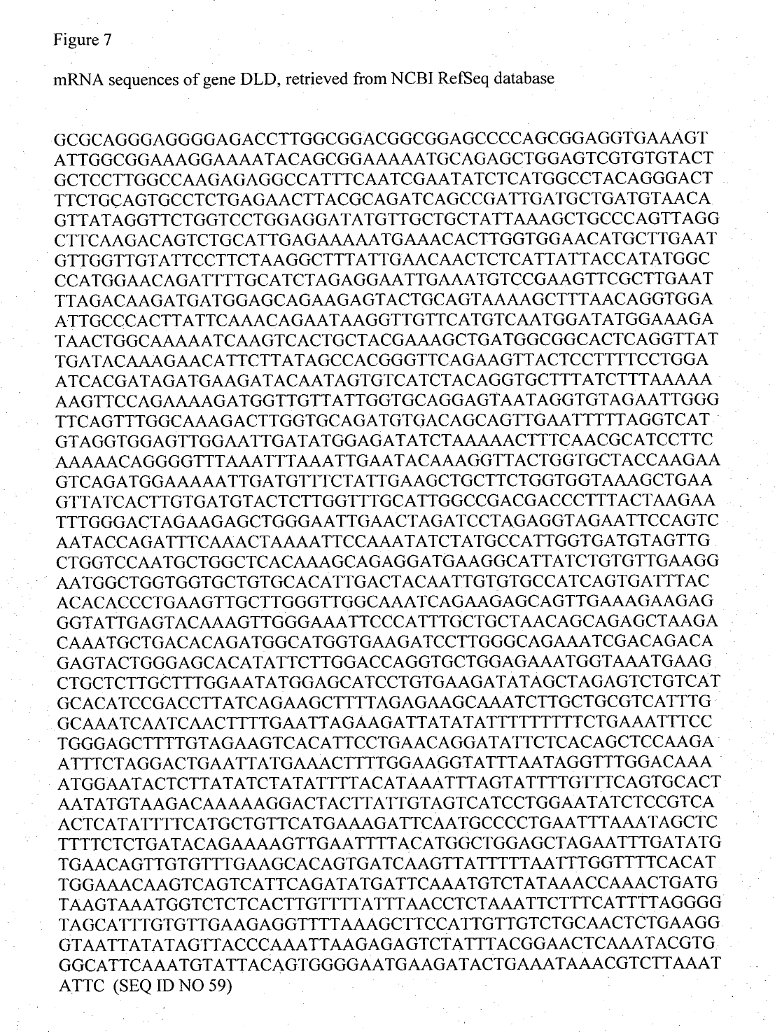

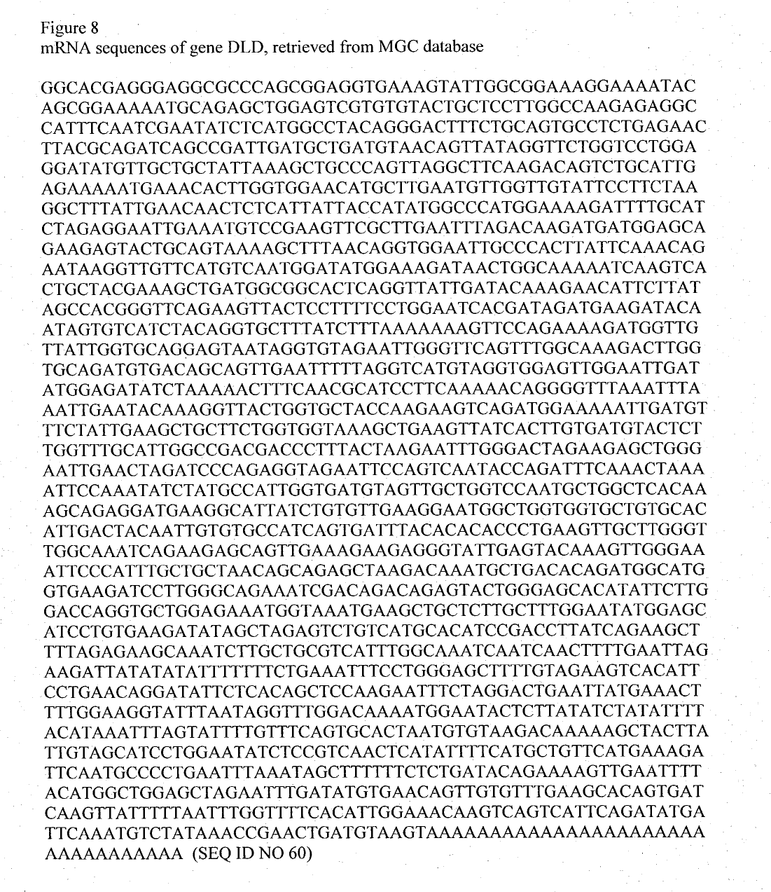

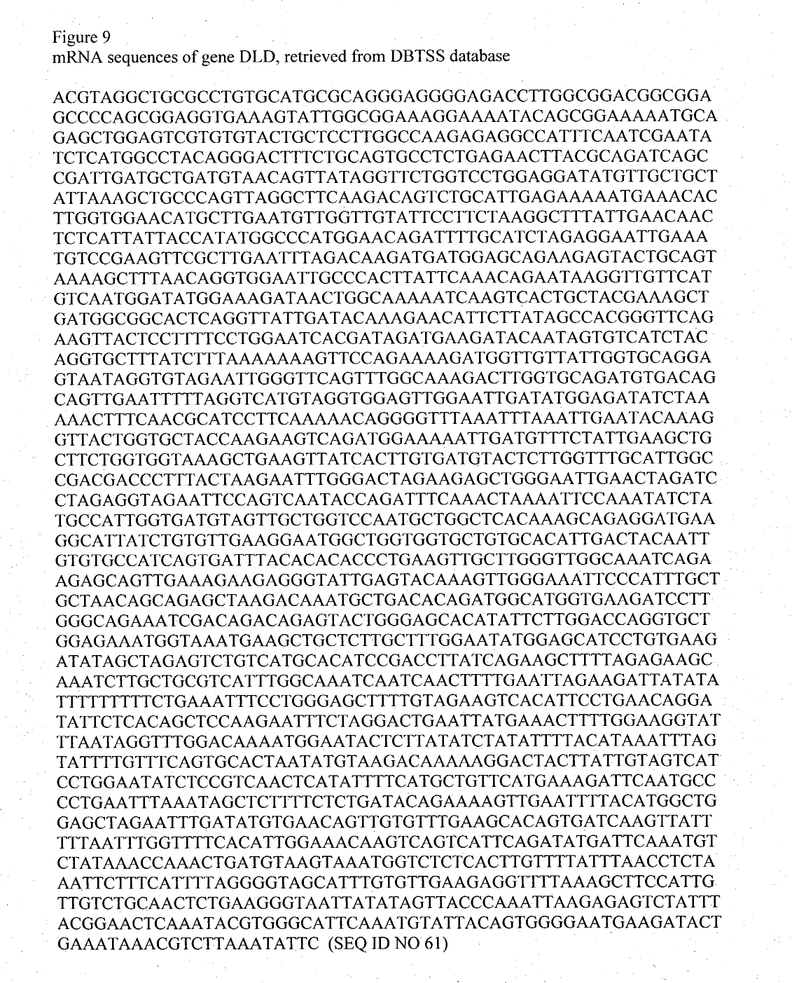

As illustrated in FIG. 2, three different versions of full-length mRNA sequences can be retrieved from NCBI database (refseq), MGC database (MGC), Japan DBTSS database (DBTSS), and the like. The completely sequences for gene DLD retrieved from refseq database is listed in FIG. 7 (SEQ ID NO 59), and the one retrieved from MGC is listed in FIG. 8 (SEQ ID NO 60), and the one retrieved from DBTSS is listed in FIG. 9 (SEQ ID NO 61).

2. The Retrieved Genes are Mapped to a Recently Updated Human Genome.

A self-developed script is used to fetch the above retrieved sequence to UCSC genome browser database to map their genomic position. The retrieved different version of gene DLD are mapped to the recently updated human genome using a tool provided by at least one of public available UCSC genome browser databases.

3. The Position of the TSS is Mapped.

The mapped positions are retrieved using self-developed script from the above referenced UCSC genome browser database. The summary result of mapping is listed in table 1. For example, the full length gene DLD sequence from NCBI refseq database was mapped to the human genome working draft (released June 2002 by UCSC genome browser) at the chromosome 7 sense strand or positive strand, starting at the chromosome position of 106015510, ending at the chromosome position of 106044308.

TABLE 1

| name | chromosome | strand | start | end |
|---|---|---|---|---|
| DLD from refseq | 7 | + | 106015510 | 106044308 |
| DLD from MGC | 7 | + | 106015541 | 106044089 |
| DLD from DBTSS | 7 | + | 106015488 | 106044308 |

4. The TSS is Mapped by Making the Most 5″ TSS of Each Gene After Comparing All Available TSS'S for the Gene.

Referring again to FIG. 2, this mapping is facilitated by using self-generated script.

For gene DLD, since it is located on “+” strand of chromosome 7. The start position 106015488 is taken as the most 5′ position for TSS of gene DLD.

5. A Genomic Sequence of a Regulatory Region for Each Retrieved Gene with the Most 5′TSS is Retrieved from the Most Updated Human Genome.

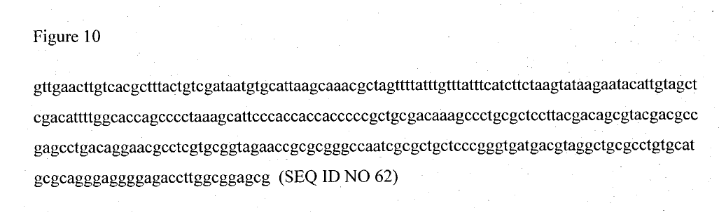

The 5′ regulatory region is the sequences upstream of the TSS and downstream of the TSS. More specifically, for gene DLD, the regulatory region or core promoter region is the sequence includes 200-300 bases upstream and the sequence about 50-100 bases downstream of the TSS. Therefore, the corresponding sequences relative to TSS of gene DLD are cut and stored with the use of self-developed scripts from at least one of the UCSC genome browser or NCBI genome database. The stored sequence for gene DLD is listed in FIG. 10 (SEQ ID NO 62).

6. The Stored Sequence for Regulatory Region of Gene DLD is Screened Using a Match Program.

The MATCH program is the sequence-analyzing tool embedded inside the licensed TRANSFAC database. The analysis is done with the proper setting for both the scores of matrix similarity and scores of core similarity in order to reduce the false positive and false negative matching during screening. The result of the screening for the regulatory region of gene DLD is shown in table 2 where the positions of identified binding sites are listed.

TABLE 2

| | | | | | |
|---|---|---|---|---|---|
| position | strand | core score | matrix score | sequences | TF name |

| | | | | | |
|---|---|---|---|---|---|
| 3 | (+) | 1 | 0.964 | tgaacttgTCACGctttactg | Pax-3 |
| | | | | (SEQ ID NO 4) | |
| 5 | (−) | 0.796 | 0.779 | aacttgtcacgCTTTActgtc | Pax-4 |
| | | | | (SEQ ID NO 5) | |
| 6 | (+) | 1 | 0.886 | acttgCACGctttactgtcg | Pax-6 |
| | | | | (SEQ ID NO 6) | |
| 6 | (+) | 0.977 | 0.761 | acttgTCACGctttactgtcg | Pax-4 |
| | | | | (SEQ ID NO 7) | |
| 24 | (+) | 0.994 | 0.972 | tCGATAatg | Lmo2 complex |
| | | | | (SEQ ID NO 8) | |
| 25 | (−) | 0.951 | 0.963 | cgataatgtgCATTAagc | Cart-1 |
| | | | | (SEQ ID NO 9) | |
| 25 | (+) | 0.951 | 0.952 | cgaTAATGtgcattaagc | Cart-1 |
| | | | | (SEQ ID NO 10) | |
| 34 | (+) | 0.896 | 0.772 | gcaTTAAGcaaa | Cdc5 |
| | | | | (SEQ ID NO 11) | |
| 48 | (+) | 1 | 0.806 | ctagtTTTATttgt | Cdx-2 |
| | | | | (SEQ ID NO 12) | |
| 50 | (−) | 0.96 | 0.937 | agtttTATTTgtttattt | FOXJ2 |
| | | | | (SEQ ID NO 13) | |
| 50 | (+) | 1 | 0.952 | agtttTATTTgttta | HNF-3 beta |
| | | | | (SEQ ID NO 14) | |
| 51 | (+) | 1 | 0.938 | gttttATTTGttt | Xvent-1 |
| | | | | (SEQ ID NO 15) | |
| 52 | (+) | 0.948 | 0.926 | ttTTATTtgttt | FOXD3 |
| | | | | (SEQ ID NO 16) | |
| 52 | (+) | 0.981 | 0.98 | tttTATTTgttta | HFH-3 |
| | | | | (SEQ ID NO 17) | |
| 54 | (−) | 1 | 0.982 | ttattTGTTTatttcatc | FOXJ2 |
| | | | | (SEQ ID NO 18) | |
| 54 | (+) | 1 | 0.926 | ttattTGTTTatttc | HNF-3 beta |
| | | | | (SEQ ID NO 19) | |
| 55 | (−) | 1 | 0.947 | tatttgTTTATttc | XFD-2 |
| | | | | (SEQ ID NO 20) | |
| 55 | (−) | 1 | 0.983 | tatttgTTTATttcat | Freac-7 |
| | | | | (SEQ ID NO 21) | |
| 55 | (−) | 1 | 0.996 | tattTGTTTat | FOXO4 |
| | | | | (SEQ ID NO 22) | |
| 55 | (+) | 0.972 | 0.97 | TATTTgtttat | HNF-3 alpha |
| | | | | (SEQ ID NO 23) | |
| 56 | (+) | 1 | 0.834 | atttgTTTATttca | Cdx-2 |
| | | | | (SEQ ID NO 24) | |
| 56 | (+) | 1 | 0.971 | attTGTTTatttc | HFH-3 |
| | | | | (SEQ ID NO 25) | |
| 56 | (+) | 1 | 0.986 | attTGTTTatttc | HFH-8 |
| | | | | (SEQ ID NO 26) | |
| 56 | (+) | 1 | 0.963 | atttGTTTAttt | HFH-1 |
| | | | | (SEQ ID NO 27) | |
| 56 | (+) | 1 | 0.958 | atTTGTTtattt | FOXD3 |
| | | | | (SEQ ID NO 28) | |
| 59 | (+) | 1 | 0.919 | TGTTTatttca | HNF-3alpha |
| | | | | (SEQ ID NO 29) | |
| 60 | (−) | 0.85 | 0.878 | gtttatttcatCTTCTaa | IRF-7 |
| | | | | (SEQ ID NO 30) | |
| 72 | (+) | 1 | 0.964 | ttctAAGTAtaa | NKX3A |
| | | | | (SEQ ID NO 31) | |
| 72 | (+) | 0.824 | 0.873 | ttctaAGTATaagaatacattgta | STAT5A |
| | | | | (SEQ ID NO 32) | (homotetramer) |
| 123 | (−) | 1 | 0.962 | agcaTTCCCacca | lk-1 |
| | | | | (SEQ ID NO 33) | |
| 123 | (−) | 1 | 0.927 | agcaTTCCCacca | lk-3 |
| | | | | (SEQ ID NO 34) | |
| 147 | (−) | 0.813 | 0.869 | gCGACAaa | E2F |
| | | | | (SEQ ID NO 35) | |
| 154 | (−) | 0.789 | 0.755 | agccctgcgctCCTTAcgaca | Pax-4 |
| | | | | (SEQ ID NO 36) | |
| 202 | (−) | 0.96 | 0.925 | gcctCGTGCg | USF |
| | | | | (SEQ ID NO 37) | |
| 222 | (+) | 1 | 0.934 | gcgggCCAATcg | CCAATbox |
| | | | | (SEQ ID NO 38) | |
| 234 | (−) | 0.788 | 0.784 | cgctgctcccgGGTGAtgacg | Pax-4 |
| | | | | (SEQ ID NO 39) | |
| 237 | (−) | 0.964 | 0.902 | tgctcccgggTGATGacgtag | Muscle initiator |
| | | | | (SEQ ID NO 40) | sequence-20 |
| 244 | (+) | 0.91 | 0.839 | gggtGATGAcgtaggctgc | v-Maf |
| | | | | (SEQ ID NO 41) | |
| 246 | (+) | 1 | 0.991 | gtgaTGACGtag | CREB |
| | | | | (SEQ ID NO 42) | |
| 248 | (+) | 1 | 0.881 | gaTGACGtaggc | ATF4 |
| | | | | (SEQ ID NO 43) | |
| 248 | (+) | 1 | 0.954 | gaTGACGtaggc | CREB |
| | | | | (SEQ ID NO 44) | |
| 250 | (+) | 0.973 | 0.951 | tgacGTAGG | TFII-I |
| | | | | (SEQ ID NO 45) | |
| 250 | (+) | 1 | 0.971 | tGACGtag | CREB |
| | | | | (SEQ ID NO 46) | |
| 277 | (+) | 1 | 0.97 | aGGGAGgg | MAZ |
| | | | | (SEQ ID NO 47) | |
| 290 | (+) | 1 | 0.999 | cTTGGCgg | E2F-1 |
| | | | | (SEQ ID NO 48) | |
| 290 | (+) | 0.964 | 0.916 | ctTGGCGg | E2F-1 |
| | | | | (SEQ ID NO 49) | |
| 290 | (+) | 0.984 | 0.897 | ctTGGCGg | E2F |
| | | | | (SEQ ID NO 50) | |

7. A Genomic or Tissue Specific Frequency of Each Binding Site is Determined.

The frequency is the existence of specific TF binding sites in regulatory regions of all the genes or tissue specific genes. After analysis of the regulatory region of all the genes, the frequency or probability of existence of TF binding sites is easy established. Some of these frequencies information are listed for gene DLD in table 3:

TABLE 3

| | | | distance | genomic |
|---|---|---|---|---|
| TF name | left position | right position | (base) to TSS | frequency |
| Pax-3 | 106015239 | 106015259 | −249 | 0.426259226 |
| Pax-4 | 106015241 | 106015261 | −247 | 0.96109025 |
| Pax-6 | 106015242 | 106015262 | −246 | 0.112003108 |
| Pax-4 | 106015242 | 106015262 | −246 | 0.96109025 |
| Lmo2complex | 106015260 | 106015268 | −228 | 0.120419526 |
| Cart-1 | 106015261 | 106015278 | −227 | 0.020134663 |
| Cart-1 | 106015261 | 106015278 | −227 | 0.020134663 |
| Cdc5 | 106015270 | 106015281 | −218 | 0.360481678 |
| Cdx-2 | 106015284 | 106015297 | −204 | 0.259031464 |
| FOXJ2 | 106015286 | 106015303 | −202 | 0.167875178 |
| HNF-3beta | 106015286 | 106015300 | −202 | 0.23688981 |
| Xvent-1 | 106015287 | 106015299 | −201 | 0.678946005 |
| HFH-3 | 106015288 | 106015300 | −200 | 0.066942898 |
| FOXD3 | 106015288 | 106015299 | −200 | 0.653632008 |
| FOXJ2 | 106015290 | 106015307 | −198 | 0.167875178 |
| HNF-3beta | 106015290 | 106015304 | −198 | 0.23688981 |
| FOXO4 | 106015291 | 106015301 | −197 | 0.10785964 |
| XFD-2 | 106015291 | 106015304 | −197 | 0.033665674 |
| Freac-7 | 106015291 | 106015306 | −197 | 0.076718892 |
| HNF-3alpha | 106015291 | 106015301 | −197 | 0.312184384 |
| HFH-1 | 106015292 | 106015303 | −196 | 0.01657387 |
| HFH-3 | 106015292 | 106015304 | −196 | 0.066942898 |
| HFH-8 | 106015292 | 106015304 | −196 | 0.020652596 |
| FOXD3 | 106015292 | 106015303 | −196 | 0.653632008 |
| Cdx-2 | 106015292 | 106015305 | −196 | 0.259031464 |
| HNF-3alpha | 106015295 | 106015305 | −193 | 0.312184384 |
| IRF-7 | 106015296 | 106015313 | −192 | 0.206914411 |
| NKX3A | 106015308 | 106015319 | −180 | 0.02233588 |
| STAT5A(homotetramer) | 106015308 | 106015331 | −180 | 0.02926324 |
| Ik-1 | 106015359 | 106015371 | −129 | 0.149682766 |
| Ik-3 | 106015359 | 106015371 | −129 | 0.019875696 |
| E2F | 106015383 | 106015390 | −105 | 0.566230739 |
| Pax-4 | 106015390 | 106015410 | −98 | 0.96109025 |
| USF | 106015438 | 106015447 | −50 | 0.781561569 |
| CCAATbox | 106015458 | 106015469 | −30 | 0.288488929 |
| Pax-4 | 106015470 | 106015490 | −18 | 0.96109025 |
| Muscleinitiator sequence-20 | 106015473 | 106015493 | −15 | 0.29004273 |
| v-Maf | 106015480 | 106015498 | −8 | 0.233458501 |
| CREB | 106015482 | 106015493 | −6 | 0.308429367 |
| CREB | 106015484 | 106015495 | −4 | 0.308429367 |
| ATF4 | 106015484 | 106015495 | −4 | 0.142172731 |
| TFII-I | 106015486 | 106015494 | −2 | 0.949177781 |
| CREB | 106015486 | 106015493 | −2 | 0.308429367 |
| MAZ | 106015513 | 106015520 | 25 | 1.118477276 |
| E2F | 106015526 | 106015533 | 38 | 0.566230739 |
| E2F-1 | 106015526 | 106015533 | 38 | 0.901268937 |
| E2F-1 | 106015526 | 106015533 | 38 | 0.901268937 |

8. A Conservation Score for Each Binding Site is Created.

The conservation scores for whole genome comparison between human and mouse are retrieved from UCSC genome browser database. The conservation score is selected to cover regions where the TF binding sites are identified. The conservation scores for the TF binding sites identified in the regulatory region of gene DLD are listed in table 4.

TABLE 4

| | | | | | |
|---|---|---|---|---|---|
| | | start | end | distance | Conservation |
| TF name | core sequences | position | position | to TSS | score |

| | | | | | |
|---|---|---|---|---|---|
| Pax-3 | tgaacttgTCACGctttactg | 106015239 | 106015259 | −249 | 0.426 |
| | (SEQ ID NO 4) | | | | |
| Pax-4 | aacttgtcacgCTTTActgtc | 106015241 | 106015261 | −247 | 0.3552 |
| | (SEQ ID NO 5) | | | | |
| Pax-6 | acttgTCACGctttactgtcg | 106015242 | 106015262 | −246 | 0.3552 |
| | (SEQ ID NO 6) | | | | |
| Pax-4 | acttgTCACGctttactgtcg | 106015242 | 106015262 | −246 | 0.3552 |
| | (SEQ ID NO 7) | | | | |
| Lmo2complex | tCGATAatg | 106015260 | 106015268 | −228 | 0.06 |
| | (SEQ ID NO 8) | | | | |
| Cart-1 | cgaTAATGtgcattaagc | 106015261 | 106015278 | −227 | 0.06 |
| | (SEQ ID NO 10) | | | | |
| Cart-1 | cgataatgtgCATTAagc | 106015261 | 106015278 | −227 | 0.06 |
| | (SEQ ID NO 9) | | | | |
| Cdc5 | gcaTTAAGcaaa | 106015270 | 106015281 | −218 | 0.064 |
| | (SEQ ID NO 11) | | | | |
| Cdx-2 | ctagtTTTATttgt | 106015284 | 106015297 | −204 | 0.11 |
| | (SEQ ID NO 12) | | | | |
| FOXJ2 | agtttTATTTgtttattt | 106015286 | 106015303 | −202 | 0.162 |
| | (SEQ ID NO 13) | | | | |
| HNF- | agtttTATTTgttta | 106015286 | 106015300 | −202 | 0.162 |
| 3beta | (SEQ ID NO 14) | | | | |
| Xvent-1 | gttttATTTGttt | 106015287 | 106015299 | −201 | 0.122666667 |
| | (SEQ ID NO 15) | | | | |
| HFH-3 | tttTATTTgttta | 106015288 | 106015300 | −200 | 0.162 |
| | (SEQ ID NO 17) | | | | |
| FOXD3 | ttTTATTtgttt | 106015288 | 106015299 | −200 | 0.122666667 |
| | (SEQ ID NO 16) | | | | |
| FOXJ2 | ttattTGTTTatttcatc | 106015290 | 106015307 | −198 | 0.286 |
| | (SEQ ID NO 18) | | | | |
| HNF- | ttattTGTTTatttc | 1060T5290 | 106015304 | −198 | 0.192 |
| 3beta | (SEQ ID NO 19) | | | | |
| FOXO4 | tattTGTTTat | 106015291 | 106015301 | −197 | 0.192 |
| | (SEQ ID NO 22) | | | | |
| XFD-2 | tatttgTTTATttc | 106015291 | 106015304 | −197 | 0.192 |
| | (SEQ ID NO 20) | | | | |
| Freac-7 | tatttgTTTATttcat | 106575291 | 106015306 | −197 | 0.286 |
| | (SEQ ID NO 21) | | | | |
| HNF- | TATTTgtttat | 106015291 | 106015301 | −197 | 0.192 |
| 3alpha | (SEQ ID NO 23) | | | | |
| HFH-1 | atttGTTTAttt | 106015292 | 106015303 | −196 | 0.192 |
| | (SEQ ID NO 27) | | | | |
| HFH-3 | attTGTTTatttc | 106015292 | 106015304 | −196 | 0.192 |
| | (SEQ ID NO 25) | | | | |
| HFH-8 | attTGTTTatttc | 106015292 | 106015304 | −196 | 0.192 |
| | (SEQ ID NO 26) | | | | |
| FOXD3 | atTTGTTtattt | 106015292 | 106015303 | −196 | 0.192 |
| | (SEQ ID NO 28) | | | | |
| Cdx-2 | atttgTTTATttca | 1060T5292 | 106015305 | −196 | 0.286 |
| | (SEQ ID NO 24) | | | | |
| HNF- | TGTTTatttca | 106015295 | 106015305 | −193 | 0.357333333 |
| 3alpha | (SEQ ID NO 29) | | | | |
| IRF-7 | gtttatttcatCTTCTaa | 106015296 | 106015313 | −192 | 0.41 |
| | (SEQ ID NO 30) | | | | |
| NKX3A | ttctAAGTAtaa | 106015308 | 106015319 | −180 | 0.624 |
| | (SEQ ID NO 31) | | | | |
| STAT5A | ttctaAGTATaagaatacattgta | 106015308 | 106015331 | −180 | 0.550666667 |
| (homotetramer) | (SEQ ID NO 32) | | | | |
| lk-1 | agcaTTCCCacca | 106015359 | 106015371 | −129 | 0.394 |
| | (SEQ ID NO 33) | | | | |
| lk-3 | agcaTTCCCacca | 106015359 | 106015371 | −129 | 0.394 |
| | (SEQ ID NO 34) | | | | |
| E2F | gCGACAaa | 106015383 | 106015390 | −105 | 0.674666667 |
| | (SEQ ID NO 35) | | | | |
| Pax-4 | agccctgcgctCCTTAcgaca | 106015390 | 106015410 | −98 | 1.2336 |
| | (SEQ ID NO 36) | | | | |
| USF | gcctCGTGCg | 106015438 | 106015447 | −50 | 0.888 |
| | (SEQ ID NO 37) | | | | |
| CCAAT- | gcgggCCAATcg | 106015458 | 106015469 | −30 | 1.136 |
| box | (SEQ ID NO 38) | | | | |
| Pax-4 | cgctgctcccgGGTGAtgacg | 106015470 | 106015490 | −18 | 1.3408 |
| | (SEQ ID NO 39) | | | | |
| Muscle | tgctcccgggTGATGacgtag | 106015473 | 106015493 | −15 | 1.3408 |
| initiator | (SEQ ID NO 40) | | | | |
| sequence-20 | | | | | |
| v-Maf | gggtGATGAcgtaggctgc | 106015480 | 106015498 | −8 | 1.356 |
| | (SEQ ID NO 41) | | | | |
| CREB | gtgaTGACGtag | 106015482 | 106015493 | −6 | 1.378666667 |
| | (SEQ ID NO 42) | | | | |
| CREB | gaTGACGtaggc | 106015484 | 106015495 | −4 | 1.356 |
| | (SEQ ID NO 44) | | | | |
| ATF4 | gaTGACGtaggc | 106015484 | 106015495 | −4 | 1.356 |
| | (SEQ ID NO 43) | | | | |
| TFII-I | tgacGTAGG | 106015486 | 106015494 | −2 | 1.544 |
| | (SEQ ID NO 45) | | | | |
| CREB | TGACGtag | 106015486 | 106015493 | −2 | 1.544 |
| | (SEQ ID NO 46) | | | | |
| MAZ | aGGGAGgg | 106015513 | 106015520 | 25 | 0.714666667 |
| | (SEQ ID NO 47) | | | | |
| E2F | ctTTGGCGg | 106015526 | 106015533 | 38 | 0.532 |
| | (SEQ ID NO 50) | | | | |
| E2F-1 | ctTGGCGg | 106015526 | 106015533 | 38 | 0.532 |
| | (SEQ ID NO 49) | | | | |
| E2F-1 | cTTGGCgg | 106015526 | 106015533 | 38 | 0.532 |
| | (SEQ ID NO 48) | | | | |

9. A Determination is Made of the Clustering of the Binding Sites and their Positions.

The adjacent or overlapped binding sites are clustered by using self-generated script and the corresponding position and TF are listed in the table 5 for the gene DLD.

TABLE 5

| | | | | |
|---|---|---|---|---|
| Cluster | | left | right | |
| ID | Core sequences | position | position | Transcription Factor(s) |

| | | | | |
|---|---|---|---|---|
| 1 | tgaacttgtcacgctttactgtcg | 106015239 | 106015281 | Cdc5; Cart-1; Cart-1; |
| | ataatgtgcattaagcaaa | | | Lmo2complex; Pax-4; |
| | (SEQ ID NO 51) | | | Pax-6; Pax-4; Pax-3; |
| 2 | ctagttttatttgtttatttcatcttc | 106015284 | 106015331 | STAT5A(homotetramer); N |
| | taagtataagaatacattgta | | | KX3A; IRF-7; |
| | (SEQ ID NO 52) | | | HNF-3 alpha; Cdx-2; HFH-3; |
| | | | | HFH-8; HFH-1; |
| | | | | FOXD3; Freac-7; XFD-2; |
| | | | | HNF-3alpha; |
| | | | | FOXO4; FOXJ2; HNF- |
| | | | | 3beta; HFH-3; |
| | | | | FOXD3; Xvent-1; |
| | | | | FOXJ2; HNF-3beta; Cdx-2; |
| 3 | agcattcccacca | 106015359 | 106015371 | lk-3; lk-1; |
| | (SEQ ID NO 53) | | | |
| 4 | gcgacaaagccctgcgctcctt | 106015383 | 106015410 | Pax-4; E2F; |
| | acgaca | | | |
| | (SEQ ID NO 54) | | | |
| 5 | gcctcgtgcg | 106015438 | 106015447 | USF; |
| | (SEQ ID NO 55) | | | |
| 6 | gcgggcaatcgcgctgctccc | 106015458 | 106015498 | TFII-I; |
| | gggtgatgacgtaggctgc | | | CREB; CREB; ATF4; |
| | (SEQ ID NO 56) | | | CREB; v-Maf; |
| | | | | Muscle initiator |
| | | | | sequence-20; Pax-4; |
| | | | | CCAATbox; |
| 7 | agggaggg | 106015513 | 105015520 | MAZ; |
| | (SEQ ID NO 57) | | | |
| 8 | cttggcgg | 106015526 | 106015533 | E2F; E2F-1; E2F-1; |
| | (SEQ ID NO 58) | | | |

10. Binding Profiles are Collected in the Database.

All the binding profiles listed above are been collected in the database. The example list of the entry for gene DLD is shown in Table 6.

TABLE 6

| | | | | | | | | |
|---|---|---|---|---|---|---|---|---|
| | | | | | | distance | | |
| | core | matrix | | left | right | (base) to | genomic | conservation |
| TF name | score | score | core sequences | position | position | TSS | frequency | score |

| | | | | | | | | |
|---|---|---|---|---|---|---|---|---|
| Pax-3 | 1 | 0.964 | tgaacttaTCACGctttactg | 106015239 | 106015259 | −249 | 0.426259226 | 0.426 |
| | | | (SEQ ID NO 63) | | | | | |
| Pax-4 | 0.796 | 0.779 | aacttgtcacgCTTTActgtc | 106015241 | 106015261 | −247 | 0.96109025 | 0.3552 |
| | | | (SEQ ID NO 5) | | | | | |
| Pax-6 | 1 | 0.886 | acttgTCACGctttactgtcg | 106015242 | 106015262 | −246 | 0.112003108 | 0.3552 |
| | | | (SEQ ID NO 6) | | | | | |
| Pax-4 | 0.977 | 0.761 | acttgTCACGctttactgtcg | 106015242 | 106015262 | −246 | 0.96109025 | 0.3552 |
| | | | (SEQ ID NO 7) | | | | | |
| Lmo2complex | 0.994 | 0.972 | tCGATAatg | 106015260 | 106015268 | −228 | 0.120419526 | 0.06 |
| | | | (SEQ ID NO 8) | | | | | |
| Cart-1 | 0.951 | 0.952 | caaTAATGtgcattaagc | 106015261 | 106015278 | −227 | 0.020134663 | 0.06 |
| | | | (SEQ ID NO 64) | | | | | |
| Cart-1 | 0.951 | 0.963 | cgataatgtgCATTAagc | 106015261 | 106015278 | −227 | 0.020134663 | 0.06 |
| | | | (SEQ ID NO 9) | | | | | |
| Cdc5 | 0.896 | 0.772 | gcaTTAAGcaaa | 106015270 | 106015281 | −218 | 0.360481678 | 0.064 |
| | | | (SEQ ID NO 11) | | | | | |
| Cdx-2 | 1 | 0.806 | ctagtTTTATttgt | 106015284 | 106015297 | −204 | 0.259031464 | 0.11 |
| | | | (SEQ ID NO 12) | | | | | |
| FOXJ2 | 0.96 | 0.937 | agtttTATTTgtttattt | 106015286 | 106015303 | −202 | 0.167875178 | 0.162 |
| | | | (SEQ ID NO 13) | | | | | |
| HNF-3beta | 1 | 0.952 | agtttTATTTgttta | 106015286 | 106015300 | −202 | 0.23688981 | 0.162 |
| | | | (SEQ ID NO 14) | | | | | |
| Xvent-1 | 1 | 0.938 | gttttATTGttt | 106015287 | 106015299 | −201 | 0.678946005 | 0.122666667 |
| | | | (SEQ ID NO 15) | | | | | |
| HFH-3 | 0.981 | 0.98 | tttTATTTgttta | 106015288 | 106015300 | −200 | 0.066942898 | 0.162 |
| | | | (SEQ ID NO 17) | | | | | |
| FOXD3 | 0.948 | 0.926 | ttTTATTtgttt | 106015288 | 106015299 | −200 | 0.653632008 | 0.122666667 |
| | | | (SEQ ID NO 16) | | | | | |
| FOXJ2 | 1 | 0.982 | ttattTGTTTatttcatc | 106015290 | 106015307 | −198 | 0.167875178 | 0.286 |
| | | | (SEQ ID NO 18) | | | | | |
| HNF-3beta | 1 | 0.926 | ttattTGTTTatttc | 106015290 | 106015304 | −198 | 0.23688981 | 0.192 |
| | | | (SEQ ID NO 19) | | | | | |
| FOX04 | 1 | 0.996 | tattTGTTTat | 106015291 | 106015301 | −197 | 0.10785964 | 0.192 |
| | | | (SEQ ID NO 22) | | | | | |
| XFD-2 | 1 | 0.947 | tatttgTTTATttc | 106015291 | 106015304 | −197 | 0.033665674 | 0.192 |
| | | | (SEQ ID NO 20) | | | | | |
| Freac-7 | 1 | 0.983 | tatttgTTTATttcat | 106015291 | 106015306 | −197 | 0.076718892 | 0.286 |
| | | | (SEQ ID NO 21) | | | | | |
| HNF-3alpha | 0.972 | 0.97 | TATTTgtttat | 106015291 | 106015301 | −197 | 0.312184384 | 0.192 |
| | | | SEQ ID NO 23) | | | | | |
| HFH-1 | 1 | 0.963 | atttGTTTAttt | 106015292 | 106015303 | −196 | 0.01657387 | 0.192 |
| | | | (SEQ ID NO 27) | | | | | |
| HFH-3 | 1 | 0.971 | attTGTTTatttc | 106015292 | 106015304 | −196 | 0.066942898 | 0.192 |
| | | | (SEQ ID NO 25) | | | | | |
| HFH-8 | 1 | 0.986 | attTGTTTatttc | 106015292 | 106015304 | −196 | 0.020652596 | 0.192 |
| | | | (SEQ ID NO 26) | | | | | |
| FOXD3 | 1 | 0.958 | atTTGTTtattt | 106015292 | 106015303 | −196 | 0.653632008 | 0.192 |
| | | | (SEQ ID NO 28) | | | | | |
| Cdx-2 | 1 | 0.834 | atttgTTTATttca | 106015292 | 106015305 | −196 | 0.259031464 | 0.286 |
| | | | (SEQ ID NO 24) | | | | | |
| HNF-3alpha | 1 | 0.919 | TGTTTatttca | 106015295 | 106015305 | −193 | 0.312184384 | 0.357333333 |
| | | | (SEQ ID NO 29) | | | | | |
| IRF-7 | 0.85 | 0.878 | gtttatttcatCTTCTaa | 106015296 | 106015313 | −192 | 0.206914411 | 0.41 |
| | | | (SEQ ID NO 30) | | | | | |
| NKX3A | 1 | 0.964 | ttctAAGTAtaa | 106015308 | 106015319 | −180 | 0.02233588 | 0.624 |
| | | | (SEQ ID NO 31) | | | | | |
| STAT5A | 0.824 | 0.873 | ttctaAGTATaagaatacatt | 106015308 | 106015331 | −180 | 0.02926324 | 0.550666667 |
| (homotetramer) | | | gta | | | | | |
| | | | (SEQ ID NO 32) | | | | | |
| lk-1 | 1 | 0.962 | agcaTTCCCacca | 106015359 | 106015371 | −129 | 0.149682766 | 0.394 |
| | | | (SEQ ID NO 33) | | | | | |
| lk-3 | 1 | 0.927 | agcaTTCCacca | 106015359 | 106015371 | −129 | 0.019875696 | 0.394 |
| | | | (SEQ ID NO 65) | | | | | |
| E2F | 0.813 | 0.869 | gCGACAaa | 106015383 | 106015390 | −105 | 0.566230739 | 0.674666667 |
| | | | (SEQ ID NO 35) | | | | | |
| Pax-4 | 0.789 | 0.755 | agccctgcgctCCTTAcgaca | 106015390 | 106015410 | −98 | 0.96109025 | 1.2336 |
| | | | (SEQ ID NO 36) | | | | | |
| USF | 0.96 | 0.925 | gcctCGTGCg | 106015438 | 106015447 | −50 | 0.781561569 | 0.888 |
| | | | (SEQ ID NO 37) | | | | | |
| CCAATbox | 1 | 0.934 | gcgggCCAATcg | 106015458 | 106015469 | −30 | 0.288488929 | 1.136 |
| | | | (SEQ ID NO 38) | | | | | |
| Pax-4 | 0.788 | 0.784 | cgctgctcccgGGTGAtgacg | 106015470 | 106015490 | −18 | 0.96109025 | 1.3408 |
| | | | (SEQ ID NO 39) | | | | | |
| Muscle | 0.964 | 0.902 | tgctcccgggTGATGacgtag | 106015473 | 106015493 | −1.5 | 0.29004273 | 1.3408 |
| initiator | | | (SEQ ID NO 40) | | | | | |
| sequence-20 | | | | | | | | |
| v-Maf | 0.91 | 0.839 | gggtGATGAcgtaggctgc | 106015480 | 106015498 | −8 | 0.233458501 | 1.356 |
| | | | (SEQ ID NO 41) | | | | | |
| CREB | 1 | 0.991 | gtgaTGACGtag | 106015482 | 106015493 | −6 | 0.308429367 | 1.378666667 |
| | | | (SEQ ID NO 42) | | | | | |
| CREB | 1 | 0.954 | gaTGACGtaggc | 106015484 | 106015495 | −4 | 0.308429367 | 1.356 |
| | | | (SEQ ID NO 44) | | | | | |
| ATF4 | 1 | 0.881 | gaTGACGtaggc | 106015484 | 106015495 | −4 | 0.142172731 | 1.356 |
| | | | (SEQ ID NO 43) | | | | | |
| TFII-I | 0.973 | 0.951 | tgacGTAGG | 106015486 | 106015494 | −2 | 0.949177781 | 1.544 |
| | | | (SEQ ID NO 45) | | | | | |
| CREB | 1 | 0.971 | TGACGtag | 106015486 | 106015493 | −2 | 0.308429367 | 1.544 |
| | | | (SEQ ID NO 46) | | | | | |
| MAZ | 1 | 0.97 | aGGGAGgg | 106015513 | 106015520 | 25 | 1.118477276 | 0.714666667 |
| | | | (SEQ ID NO 47) | | | | | |
| E2F | 0.984 | 0.897 | ctTGGCGg | 106015526 | 106015533 | 38 | 0.566230739 | 0.532 |
| | | | (SEQ ID NO 50) | | | | | |
| E2F-1 | 0.964 | 0.916 | cTGGCGg | 106015526 | 106015533 | 38 | 0.901268937 | 0.532 |
| | | | (SEQ ID NO 49) | | | | | |
| E2F-1 | 1 | 0.999 | cTTGGCgg | 106015526 | 106015533 | 38 | 0.901268937 | 0.532 |
| | | | (SEQ ID NO 48) | | | | | |

11. The Database is Searchable by Gene Identifiers.

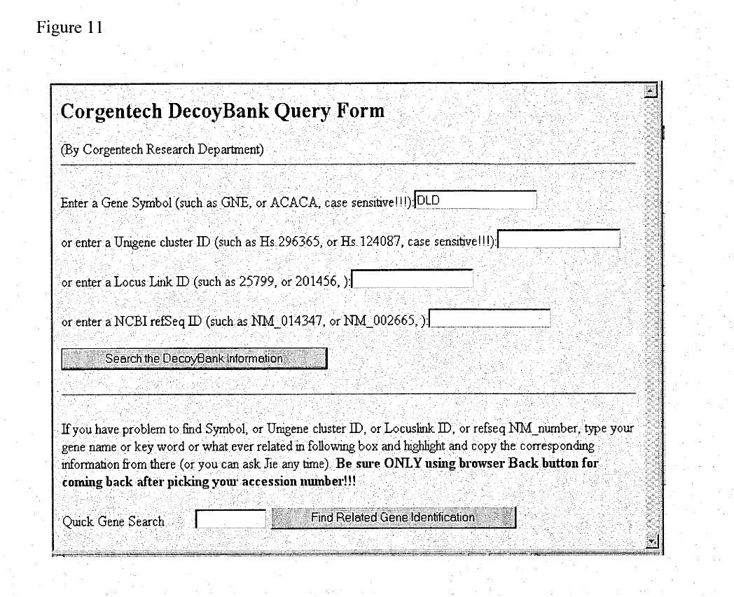

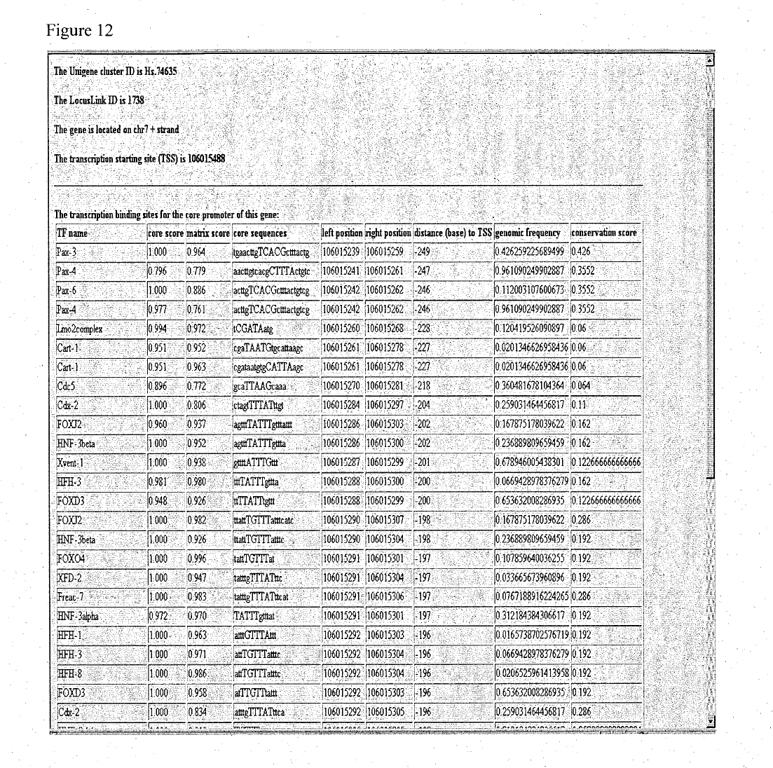

FIG. 11 illustrates a screen shot of a query form that can be used with the database. FIG. 12 illustrates a screen shot of a database query result.

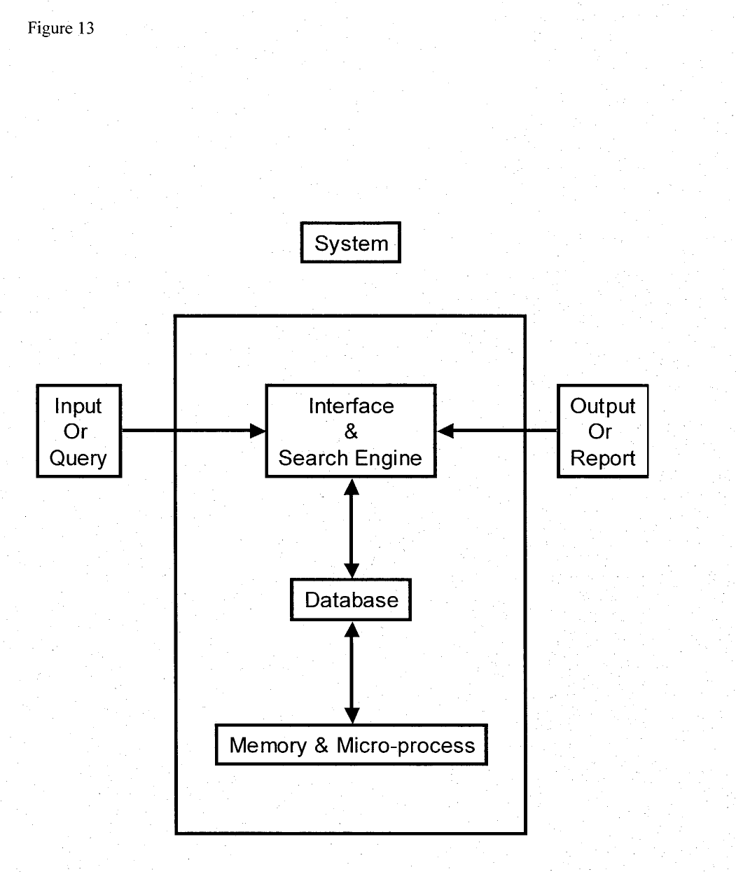

As illustrated in FIG. 13, another embodiment of the present invention is a computer implemented system for displaying the profiled regulatory factor binding sites. The system includes the database, a user interface that includes one or more selectable user inputs, an input device operable by a user, and a display for displaying at least one output in response to the profiled identified binding sites.

Examples of outputs include but are not limited to, gene name, identifier, identified TF binding site, TF names, genomic positions, length, distance, conservation score, binding scores, frequencies information, and binding sites sequences. Examples of inputs includes, the gene identifiers, such as gene symbols, unigene cluster ID, or locuslink ID, and the like.

The system also includes a memory, a microprocessor, data files, scripts, supporting available software, including but not limited to MS windows, red hat linux, Apache HTTP sever, Perl compiler program, and the like.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. A method for profiling regulatory factor binding sites;
locating a complete and most 5′ full-length gene for mapping gene regulatory regions;
retrieving genomic sequences of gene regulatory regions;
screening DNA sequence information for each retrieved gene regulatory region to identify putative regulatory factor binding sites; and
profiling the putative regulatory factor binding sites.

2. The method of claim 1, wherein mapping includes retrieving full-length genes to provide sequences information for retrieved genes.

3. The method of claim 2, wherein mapping includes, mapping the retrieved genes to a recently updated human genome.

4. The method of claim 3, wherein the retrieved genes are mapped to the recently updated human genome using a tool provided by at least one of public available UCSC genome browser databases and self-developed scripts.

5. The method of claim 3, wherein the transcription start site (TSS) is mapped.

6. The method of claim 5, wherein the TSS is mapped by taking the most 5′ TSS of each gene after comparing all available TSS's for the gene.

7. The method of claim 1, wherein a genomic sequence of a regulatory region for each retrieved gene with the most 5′ TSS is retrieved from the most updated human genome.

8. The method of claim 7, wherein the 5′ regulatory region is the sequences located upstream of the TSS and downstream of the TSS.

9. The method of claim 1, wherein a retrieved sequence of a gene regulatory region is the core promoter region.

10. The method of claim 9, wherein the core promoter region is includes 200-300 bases upstream and the sequence about 50-100 bases downstream of the TSS.

11. The method of claim 5, wherein a genomic sequence of a gene is the upstream enhancer region.

12. The method of claim 3, wherein a genomic sequence of a gene regulatory region is a downstream regulatory region.

13. The method of claim 7, further comprising:
cutting and storing the corresponding sequences relative to TSS.

14. The method of claim 13, wherein the corresponding sequences relative to TSS are cut and stored with the use of self-developed scripts from at least one of the UCSC genome browser or NCBI genome database.

15. The method of claim 1, wherein the DNA sequence information is screened using a MATCH program or the similar Position Weighted Matrix Programs for motif searching.

16. The method of claim 1, wherein the DNA sequence information screening includes selecting the TF matrix, scores of matrix similarity and scores of core similarity.

17. The method of claim 1, wherein cut-off is applied to reduce the false positive and false negative matching during screening.

18. The method of claim 1, further comprising:
determining at least one of a genomic or tissue-specific frequency of each binding site.

19. The method of claim 1, wherein the frequency is the existence of specific TF binding sites in regulatory regions of all the genes.

20. The method of claim 1, wherein the frequency is the existence of specific TF binding sites in regulatory regions of tissue specific genes.

21. The method of claim 16, further comprising:
creating a conservation score for each binding site.

22. The method of claim 17, wherein the conservation score is selected to cover regions where the TF binding sites are identified.

23. The method of claim 17, further comprising:
determining a position of each binding site.

24. The method of claim 23, wherein the position is based on a human genome working draft.

25. The method of claim 24, wherein the position is a converted position in a human genome working draft.

26. The method of claim 23, wherein the genome position of a start and end is determined.

27. The method of claim 23, further comprising:
determining a distance of each binding site to the TSS.

28. The method of claim 27, wherein the distance is relative to a number of bases between a binding site and the TSS

29. The method of claim 27, further comprising:
determining a length of each binding site.

30. The method of claim 29, further comprising:
determining sequence information about regions adjacent to the binding site.

31. The method of claim 30, further comprising:
determining co-existence information of other binding sites.

32. The method of claim 31, further comprising:
determining cluster of the binding sites and their positions.

33. The method of claim 1, further comprising:
collecting the binding profiles in a database.

34. The method of claim 33, wherein the database includes TF binding profiles for the regulatory region of each gene.

35. The method of claim 33, wherein the database is searchable by gene identifiers.

36. The method of claim 35, wherein the gene identifiers are selected from the NCBI database.

37. The method of claim 36, wherein the NCBI database includes at least one of Unigene Cluster ID, LoucsLink ID and international approved gene symbols.

38. The method of claim 35, wherein the database includes genomic frequencies information for TF.

39. The database of claim 38, wherein the database is sortable by at least one of TF name and TF frequencies

40. The method of claim 39, wherein the TF frequencies include genome frequencies and tissue specific frequencies.

41. The method of claim 33, further comprising:
retrieving information from the database for biomedical research.

42. The method of claim 33, further comprising:
retrieving information from the database for pre-clinical development.

43. The method of claim 33, further comprising:
retrieving information from the database for drug screening applications.

44. The method of claim 33, further comprising:
retrieving information from the database for target discovering and target validation.

45. The method of claim 33, further comprising:
retrieving information from the database for profiling of a regulatory region.

46. The method of claim 33, further comprising:
retrieving information from the database for building the genome or tissue wide connections between regulatory profilings of different genes.

47. The method of claim 33, further comprising:
retrieving information from the database for understanding the genome or tissue background of various known transcription profiling understanding the genome or tissue background of various known transcription profiling.

48. A method for profiling identified binding sites, comprising:
providing a database that includes profiled identified binding sites for known genes; and
applying probability mapping to the profiled binding sites.

49. The method of claim 48, wherein the database includes TF binding profiles for the regulatory region of each gene.

50. The method of claim 48, wherein the database is searchable by gene identifiers.

51. The method of claim 50, wherein the gene identifiers are selected from the NCBI database.

52. The method of claim 51, wherein the NCBI database includes at least one of Unigene Cluster ID, LoucsLink ID and international approved gene symbols.

53. The method of claim 51, wherein the database includes genomic frequencies information for vertebrate transcription regulatory factors.

54. The method of claim 53, wherein the database is sortable by at least one of TF name and TF frequencies

55. The method of claim 54, wherein the TF frequencies include genome frequencies and tissue specific frequencies.

56. The method of claim 48, further comprising:
retrieving information from the database for biomedical research.

57. The method of claim 48, further comprising:
retrieving information from the database for pre-clinical development.

58. The method of claim 48, further comprising:
retrieving information from the database for drug screening applications.

59. The method of claim 48, further comprising:
retrieving information from the database for target discovering and target validation.

60. The method of claim 48, further comprising:
retrieving information from the database for profiling of a regulatory region.

61. The method of claim 48, further comprising:
retrieving information from the database for building the genome or tissue wide connections between regulatory profilings of different genes.

62. The method of claim 48, further comprising:
retrieving information from the database for understanding the genome or tissue background of various known transcription profiling understanding the genome or tissue background of various known transcription profiling.

63. A data structure tangibly stored on a computer readable medium, comprising:
a database that includes profiled identified binding sites, the profiled identified binding sites being created by screening DNA sequence information for gene regulatory regions, and wherein the database is searchable by gene identifiers.

64. The data structure of claim 63, wherein the gene identifiers are selected from the NCBI GeneBank identifiers.

65. The method of claim 64, wherein the NCBI database includes at least one of Unigene Cluster ID, LoucsLink ID and international approved gene symbols.

66. The data structure of claim 63, wherein the database includes TF binding profiles for the regulatory region of each gene.

67. The data structure of claim 63, wherein the database includes genomic frequencies information for vertebrate transcription regulatory factors.

68. The database of claim 63, wherein the database is sortable by at least one of TF name and TF frequencies

69. The data structure of claim 68, wherein the TF frequencies include genome frequencies and tissue specific frequencies.

70. The data structure of claim 63, wherein the database includes information for biomedical research.

71. The data structure of claim 63, wherein the database includes information for pre-clinical development.

72. The data structure of claim 63, wherein the database includes information for drug screening applications.

73. The data structure of claim 63, wherein the database includes information for target discovering and target validation.

74. The data structure of claim 63, wherein the database includes information for profiling of a regulatory region.

75. The data structure of claim 63, wherein the database includes information for building the genome or tissue wide connections between regulatory profilings of different genes.

76. The data structure of claim 63, wherein the database includes information for understanding the genome or tissue background of various known transcription profiling understanding the genome or tissue background of various known transcription profiling.

77. A computer implemented system for profiling regulatory factor binding sites, comprising:
a database that includes profiled identified binding sites, the profiled identified binding sites being created by screening DNA sequence information for gene regulatory regions, and wherein the database is searchable by gene identifiers;
a user interface that includes one or more selectable user inputs;
an input device operable by a user; and
a display for displaying at least one output in response to the profiled identified binding sites.

78. The system of claim 77, wherein the gene identifiers are selected from the NCBI GeneBank identifiers.

79. The system of claim 78, wherein the NCBI database includes at least one of Unigene Cluster ID, LoucsLink ID and international approved gene symbols.

80. The system of claim 77, wherein the database includes TF binding profiles for the regulatory region of each gene.

81. The system of claim 77, wherein the database includes genomic frequencies information for vertebrate transcription regulatory factors.

82. The system of claim 77, wherein the database is sortable by at least one of TF name and TF frequencies

83. The system of claim 68, wherein the TF frequencies include genome frequencies and tissue specific frequencies.

84. The system of claim 77, wherein the database includes information for biomedical research.

85. The system of claim 77, wherein the database includes information for pre-clinical development.

86. The system of claim 77, wherein the database includes information for drug screening applications.

87. The system of claim 77, wherein the database includes information for target discovering and target validation.

88. The system of claim 77, wherein the database includes information for profiling of a regulatory region.

89. The system of claim 77, wherein the database includes information for building the genome or tissue wide connections between regulatory profilings of different genes.

90. The system of claim 77, wherein the database includes information for understanding the genome or tissue background of various known transcription profiling understanding the genome or tissue background of various known transcription profiling.

91. The system of claim 77, wherein the at least one output includes at least include one of, a gene name, an identifier, an identified TF binding site, TF names, genomic positions, length, distance, conservation score, binding scores, frequencies information, and binding sites sequences.

92. The system of claim 77, further comprising:
a memory; and
a microprocessor
